SEQUENCE LISTING (45 sequences)

SEQ ID NO: 1 - protein, 19 aa, Artificial Sequence
  JNK peptide inhibitor
DQSRPVQPFLNLTTPRKPR

SEQ ID NO: 2 - protein, 31 aa, Artificial Sequence
  JNK peptide inhibitor
DQSRPVQPFLNLTTPRKPRPPRRRQRRKKRG

SEQ ID NO: 3 - protein, 31 aa, Artificial Sequence
  JNK peptide inhibitor
GRKKRRQRRRPPRPKRPTTLNLFPQVPRSQD

SEQ ID NO: 4 - protein, 19 aa, Artificial Sequence
  JNK peptide inhibitor
RPKRPTTLNLFPQVPRSQD

SEQ ID NO: 5 - protein, 12 aa, Artificial Sequence
  protein transduction domain
GRKKRRQRRRPP

SEQ ID NO: 6 - protein, 12 aa, Artificial Sequence
  protein transduction domain
PPRRRQRRKKRG

SEQ ID NO: 7 - protein, 16 aa, Artificial Sequence
  protein transduction domain
RQIKIWFQNRRMKWKK

SEQ ID NO: 8 - protein, 7 aa, Artificial Sequence
  protein transduction domain
RRMKWKK

SEQ ID NO: 9 - protein, 18 aa, Artificial Sequence
  protein transduction domain
RGGRLSYSRRRFSTSTGR

SEQ ID NO: 10 - protein, 10 aa, Artificial Sequence
  protein transduction domain
RRLSYSRRRF

SEQ ID NO: 11 - protein, 17 aa, Artificial Sequence
  protein transduction domain
RGGRLAYLRRRWAVLGR

SEQ ID NO: 12 - protein, 8 aa, Artificial Sequence
  protein transduction domain
RRRRRRRR

SEQ ID NO: 13 - protein, 23 aa, Artificial Sequence
  JNK peptide inhibitor
DTYRPKRPTTLNLFPQVPRSQDT

SEQ ID NO: 14 - protein, 35 aa, Artificial Sequence
  JNK peptide inhibitor
GRKKRRQRRRPPDTYRPKRPTTLNLFPQVPRSQDT

SEQ ID NO: 15 - protein, 23 aa, Artificial Sequence
  JNK peptide inhibitor
TDQSRPVQPFLNLTTPRKPRYTD

SEQ ID NO: 16 - protein, 35 aa, Artificial Sequence
  JNK peptide inhibitor
TDQSRPVQPFLNLTTPRKPRYTDPPRRRQRRKKRG

SEQ ID NO: 17 - protein, 18 aa, Artificial Sequence
  JNK peptide inhibitor
HKHRPTTLRLTTLGAQDS

SEQ ID NO: 18 - protein, 30 aa, Artificial Sequence
  JNK peptide inhibitor
GRKKRRQRRRPPHKHRPTTLRLTTLGAQDS

SEQ ID NO: 19 - protein, 18 aa, Artificial Sequence
  JNK peptide inhibitor
SDQAGLTTLRLTTPRHKH

SEQ ID NO: 20 - protein, 30 aa, Artificial Sequence
  JNK peptide inhibitor
SDQAGLTTLRLTTPRHKHPPRRRQRRKKRG

SEQ ID NO: 21 - protein, 11 aa, Artificial Sequence
  JNK peptide inhibitor
RPKRPTTLNLF

SEQ ID NO: 22 - protein, 23 aa, Artificial Sequence
  JNK peptide inhibitor
GRKKRRQRRRPPRPKRPTTLNLF

SEQ ID NO: 23 - protein, 11 aa, Artificial Sequence
  JNK peptide inhibitor
FLNLTTPRKPR

SEQ ID NO: 24 - protein, 23 aa, Artificial Sequence
  JNK peptide inhibitor
FLNLTTPRKPRPPRRRQRRKKRG

SEQ ID NO: 25 - protein, 11 aa, Artificial Sequence
  JNK peptide inhibitor
RPKRPKTLNLF

SEQ ID NO: 26 - protein, 23 aa, Artificial Sequence
  JNK peptide inhibitor
GRKKRRQRRRPPRPKRPKTLNLF

SEQ ID NO: 27 - protein, 11 aa, Artificial Sequence
  JNK peptide inhibitor
FLNLTKPRKPR

SEQ ID NO: 28 - protein, 23 aa, Artificial Sequence
  JNK peptide inhibitor
FLNLTKPRKPRPPRRRQRRKKRG

SEQ ID NO: 29 - protein, 11 aa, Artificial Sequence
  JNK peptide inhibitor
RPKRPTFLNLF

SEQ ID NO: 30 - protein, 23 aa, Artificial Sequence
  JNK peptide inhibitor
GRKKRRQRRRPPRPKRPTFLNLF

SEQ ID NO: 31 - protein, 11 aa, Artificial Sequence
  JNK peptide inhibitor
FLNLFTPRKPR

SEQ ID NO: 32 - protein, 23 aa, Artificial Sequence
  JNK peptide inhibitor
FLNLFTPRKPRPPRRRQRRKKRG

SEQ ID NO: 33 - protein, 11 aa, Artificial Sequence
  JNK peptide inhibitor
RPKRPTSLNLF

SEQ ID NO: 34 - protein, 23 aa, Artificial Sequence
  JNK peptide inhibitor
GRKKRRQRRRPPRPKRPTSLNLF

SEQ ID NO: 35 - protein, 11 aa, Artificial Sequence
  JNK peptide inhibitor
FLNLSTPRKPR

SEQ ID NO: 36 - protein, 23 aa, Artificial Sequence
  JNK peptide inhibitor
FLNLSTPRKPRPPRRRQRRKKRG

SEQ ID NO: 37 - protein, 11 aa, Artificial Sequence
  JNK peptide inhibitor
RPKRPTTLNLD

SEQ ID NO: 38 - protein, 23 aa, Artificial Sequence
  JNK peptide inhibitor
GRKKRRQRRRPPRPKRPTTLNLD

SEQ ID NO: 39 - protein, 11 aa, Artificial Sequence
  JNK peptide inhibitor
DLNLTTPRKPR

SEQ ID NO: 40 - protein, 23 aa, Artificial Sequence
  JNK peptide inhibitor
DLNLTTPRKPRPPRRRQRRKKRG

SEQ ID NO: 41 - protein, 10 aa, Artificial Sequence
  JNK peptide inhibitor
PKRPTTLNLF

SEQ ID NO: 42 - protein, 22 aa, Artificial Sequence
  JNK peptide inhibitor
GRKKRRQRRRPPPKRPTTLNLF

SEQ ID NO: 43 - protein, 10 aa, Artificial Sequence
  JNK peptide inhibitor
FLNLTTPRKP

SEQ ID NO: 44 - protein, 22 aa, Artificial Sequence
  JNK peptide inhibitor
FLNLTTPRKPPPRRRQRRKKRG

SEQ ID NO: 45 - protein, 36 aa, Artificial Sequence
  JNK peptide inhibitor
YGRKKRRQRRRILKQSMTLNLADPVGSLKPHLRAKN

(19) United States
(12) Patent Application Publication
(10) Pub. No.: US 2015/0374779 A1
(43) Pub. Date: Dec. 31, 2015

(54) PHARMACOLOGIC TREATMENTS OF MENIÉRE'S DISEASE

(71) Applicant: Auris Medical AG, Basel (CH)

(72) Inventor: Thomas Meyer, Zuchwil (CH)

(21) Appl. No.: 14/751,885

(22) Filed: Jun. 26, 2015

Related U.S. Application Data
(60) Provisional application No. 62/017,624, filed on Jun. 26, 2014.

(51) Int. Cl. A61K 38/16; A61K 45/06; A61K 47/36; A61K 38/10; A61K 9/00

(52) U.S. Cl. CPC A61K 38/16; A61K 38/10; A61K 9/0046; A61K 9/0019; A61K 47/36; A61K 45/06

(57) ABSTRACT

The invention relates to pharmaceutical compositions and methods for treating Meniére's Disease. In particular, the invention provides a method for treating Meniére's Disease in a subject in need thereof by administering a pharmaceutical composition comprising a peptide inhibitor of c-Jun N-terminal kinase.

### CROSS-REFERENCE TO RELATED APPLICATIONS

This application claims priority to U.S. Provisional Application Ser. No. 62/017,624, filed Jun. 26, 2014, which is herein incorporated by reference in its entirety for all purposes.

### FIELD OF THE INVENTION

The present invention relates to the treatment of inner ear disorders, such as Meniére's Disease. More precisely, the present invention relates to compounds, pharmaceutical compositions and methods for ameliorating, treating, and/or preventing Meniére's Disease.

The pharmaceutical compositions may comprise an inhibitor of c-Jun N-terminal kinase (JNK).

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: AURS_{—}006_{—}01 US_SeqList_ST25.txt, date recorded: Jun. 22, 2015, file size: 17 kilobytes).

### BACKGROUND OF THE INVENTION

Meniére's Disease (MD) is a syndrome with various fluctuating symptoms including, but not limited to vertigo, dizziness, tinnitus, hearing loss and the sensation of pressure or pain in the affected ear. It usually breaks out suddenly, and can arise daily or as infrequently as once a year. Durations and intensities of the discomforts vary from patient to patient. Patients have sudden attacks of vertigo that usually last for 1 to 6 h but that can (rarely) last up to 24 h, usually with nausea and vomiting. Accompanying symptoms include diaphoresis, diarrhea, and gait unsteadiness (Merck Manual). Tinnitus in the affected ear may be constant or intermittent, buzzing or roaring; it is not related to position or motion. Hearing impairment, typically affecting low frequencies, may follow.

Before and during an MD episode, most patients sense fullness or pressure in the affected ear. Vertigo tends to be the most debilitating symptom of MD, especially since it can set in with no or little prior warning. During the early stages, symptoms remit between episodes; symptom-free interludes may last >1 yr. In a patient survey, the time between the first and second vertigo attack was days (25.3%) or weeks (23.8%) in most, but was >1 year in ˜15% (Derebery and Berliner, 2005). At its worst, 69% of patients had >2 spells/month and 12% reported ≧20 spells/month, with 40.8% greatly disabled. As MD progresses, hearing impairment persists and gradually worsens, and tinnitus may be constant. However, not all symptoms must be present to confirm the diagnosis. In a majority of patients, only one ear is affected; MD may begin in one ear and become bilateral as the disorder progresses.

Depending on their intensity, MD symptoms may be just a nuisance for patients or they may negatively affect their quality of life, making it impossible to perform normal activities of daily living. Other conditions can present themselves with Meniére's-like symptoms, such as, syphilis, Cogan's syndrome, autoimmune inner ear disease, perilymph fistula, multiple sclerosis, acoustic neuroma, and both hypo- and hyperthyroidism. According to the National Institute on Deafness and Other Communication Disorders, there are 615,000 patients with MD in the United States, with 45,500 new cases diagnosed per year.

To date, there is no cure for MD and patients are treated symptomatically. Although these treatments are reported to alleviate vertigo or dizziness, they do not address the underlying cause. About 70% of MD patients are estimated to respond to conservative treatment with low-salt diet, diuretics and symptom suppressants; however 30% get progressively worse and often require surgical therapy (Gates, 2007). In case of intractable vertigo, endolymphatic sac surgery, vestibular neurectomy, or chemical ablation of vestibular hair cells with ototoxic aminoglycosides (e.g. gentamicin) is performed. While the frequency and severity of vertigo tends to decline over time, and several treatment options are available, no such options have been available for the symptoms of progressive inner ear hearing loss and persistent tinnitus.

Overall, many existing or proposed treatments for MD target only endolymphatic hydrops (EH), although it has been established that EH is not the cause, but rather an epiphenomenon of MD. Many approaches target only vertigo control, but not progressive hearing loss or tinnitus, and many have significant side effects when given systemically over an extended period of time. Local administration of drugs to the inner ear, on the other hand, faces the challenge of reaching sufficient drug concentration in the apical region of the cochlea, which is of key interest in MD. It is well known in the art that certain drugs can be delivered effectively via the round window or oval window membranes into the basal part of the cochlea and the vestibule, but that achieving desired therapeutic concentrations in the apical region of the cochlea is much more difficult (Salt and Plontke, 2009).

In the light of the above and the lack of effective treatments for Meniére's Disease, there is a great need for a pharmacologic treatment that could provide relief for patients by, e.g., attenuating the degree and/or frequency of vertigo attacks, protecting against or attenuating progressive sensorineural hearing loss and attenuating or suppressing tinnitus.

### SUMMARY OF THE INVENTION

The present invention provides methods of treating Meniére's Disease in a human. In some embodiments, the methods comprise administering to the human a pharmaceutical composition comprising a therapeutically effective amount of an inhibitor of c-Jun N-terminal kinase (JNK). In some embodiments, the inhibitor is a peptide inhibitor or a pharmaceutically acceptable salt thereof.

In some embodiments, the peptide inhibitor is no more than 50 amino acids in length. In some embodiments, the peptide inhibitor comprises a sequence that is at least 80% identical to the sequence of any one of SEQ ID NOs: 1 to 4 and 13 to 45.

In some embodiments, the peptide inhibitor comprises a sequence that is at least 90% identical to DQSRPVQPFLNLTTPRKPR (SEQ ID NO: 1) or RPKRPTTLNLFPQVPRSQD (SEQ ID NO: 4).

In some embodiments, the peptide inhibitor comprises or consists of the sequence of DQSRPVQPFLNLTTPRKPRPPRRRQRRKKRG (SEQ ID NO: 2) or GRKKRRQRRRPPRPKRPTTLNLFPQVPRSQD (SEQ ID NO: 3).

In some embodiments, all of the chiral amino acids in the peptide inhibitor are in the D configuration. In some embodiments, all of the chiral amino acids in the peptide inhibitor are in the L configuration.

In some embodiments, the pharmaceutical composition ameliorates, treats or prevents one or more symptoms resulting from MD. In some embodiments, the pharmaceutical composition decreases or completely resolves one or more symptoms resulting from MD. In some embodiments, the pharmaceutical composition attenuates the severity, duration and/or frequency of one or more symptoms resulting from MD. In some embodiments, the pharmaceutical composition stabilizes the symptoms (e.g., preventing or delaying the worsening of the symptoms). In some embodiments, the pharmaceutical composition delays or slows down the progression of the symptoms. In some embodiments, the pharmaceutical composition ameliorates the disease state, decreases the dose of one or more other medications required to treat the disease, and/or increases the quality of life, etc. In some embodiments, a pharmaceutical composition of the present invention reduces the severity, duration, and/or frequency of at least two symptoms of the Meniére's Disease.

In some embodiments, a symptom of MD is selected from the group consisting of vertigo, dizziness, tinnitus, hearing loss and the sensation of pressure or pain in the affected ear. In some embodiments, the hearing loss is progressive hearing loss.

In some embodiments, the symptoms of Meniére's Disease result from idiopathic endolymphatic hydrops. In some embodiments, the symptoms of Meniére's Disease result from viral infections of sensory cells in the inner ear. In some embodiments the symptoms of Meniére's Disease result from atrial ischemia.

The pharmaceutical composition can be administered by a suitable method. In some embodiments, the pharmaceutical composition is administered locally to the round window membrane or oval window of the ear. In some embodiments, the pharmaceutical composition is administered by an intratympanic injection. In some embodiments, the pharmaceutical composition is administered systemically.

In some embodiments, a single dose or repeated doses are administered to the human. In some embodiments, the pharmaceutical composition is administered to the human having two, three, or more symptoms of Meniére's Disease.

In some embodiments, the pharmaceutical composition is administered to the human within the same day, or one, two, three, or more days following an attack of one or more symptoms of Meniére's Disease. In some embodiments, the pharmaceutical composition is administered to the human when there are one or more prodromes of MD in the human before the attack comes.

In some embodiments, the human has, or is diagnosed to have, or has risk to have progressive hearing loss in accordance with the Meniére's guidelines of the American Academy of Otolaryngology.

In some embodiments, the pharmaceutical composition is a gel. In some embodiments, the pharmaceutical composition is an implant.

In some embodiments, the pharmaceutical composition is used in a single treatment. In some embodiments, the pharmaceutical composition is used for recurrent treatment of Meniére's Disease. In some embodiments, the pharmaceutical composition is used for continuous treatment of Meniére's Disease.

In some embodiments, the pharmaceutical composition comprises polymers. In some embodiments, the polymer is hyaluronic acid. In some embodiments, the pharmaceutical composition comprises about 0.5% to about 2% of hyaluronic acid.

In some embodiments, the pharmaceutical composition comprises a buffer. In some embodiments, the buffer provides a PH from about 6.0 to about 7.4. In some embodiments, the buffer is a phosphate buffer or a citrate buffer.

In some embodiments, the pharmaceutical composition comprises about 1 to about 500 μM of a peptide inhibitor of c-Jun N-terminal kinase (JNK) or a pharmaceutically acceptable salt thereof. In some embodiments, the pharmaceutical composition comprises about 50 to about 150 μM of the inhibitor. In some embodiments, the pharmaceutical composition comprises about 100 μM of the inhibitor.

In some embodiments, the pharmaceutical composition is administered in multiple doses.

In some embodiments, the pharmaceutical composition is combined with one or more other treatments. In some embodiments, the other treatment comprises administration of an antiviral, diuretic, antihistamine and/or antiemetic agent.

In some embodiments, the treatment provides statistically significant therapeutic effect for the treatment of Meniére's Disease.

The present invention also provides methods of attenuating long-term outcomes of Meniére's Disease. In some embodiments, the methods comprise administering to the human a pharmaceutical composition of the present invention to a human diagnosed to have MD, or a human suspected to have MD. In some embodiments, at least one long-term outcome of Meniére's Disease is progressive hearing loss, such as progressive sensorineural hearing loss.

By protecting sensory cells in the stress-injured cochlea, pharmaceutical compositions of the present invention are effective in attenuating one or more symptoms and long-term outcomes of Meniére's Disease, such as progressive sensorineural hearing loss.

### BRIEF DESCRIPTION OF THE DRAWINGS

FIG. 1 shows a schematic drawing of the inner ear and its major components.

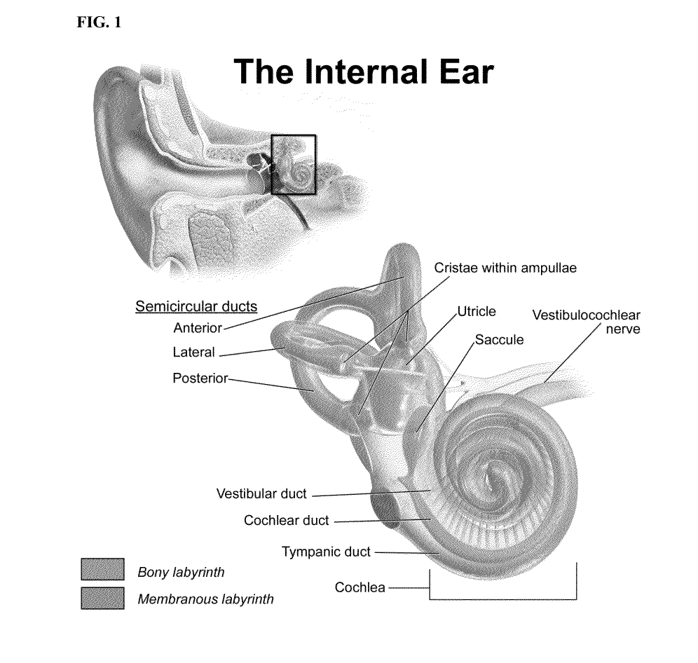

FIG. 2 shows gene expression by Western blot for the early viral protein ICP8 and the late viral protein gC in ATRA-differentiated SH-SY5Y human neuronal cells or fetal rat cortex cells (ICP8 only) which were either mock infected of infected with wild type HSV-1 (KOS). Cell monolayers were pretreated either with DMSO (DM, vehicle control), or the JNK inhibitors D-JNKI-1 (AM, 10 μM) or SP600125 (SP, 40 μM), then infected in the presence of drug for 60 minutes and finally in the presence of drug until the time of harvest, 8 hours post infection. Western blot for tubulin served as a loading control.

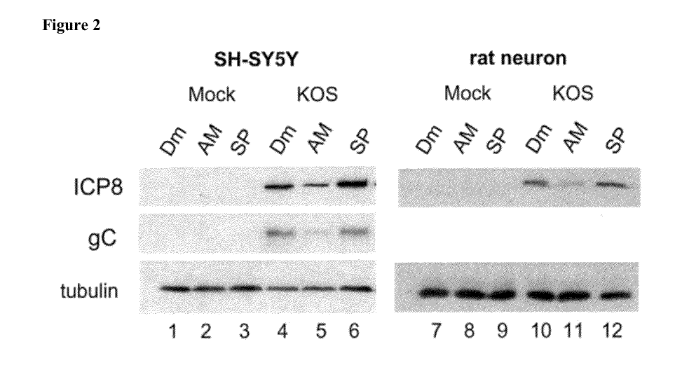

FIG. 3 shows gene expression by Western blot for the immediate-early viral protein ICP27 and the late viral protein Us11 in ATRA-differentiated SH-SY5Y human neuronal cells or fetal rat cortex cell which were either mock infected of infected with wild type HSV-1 (KOS). Cell monolayers were pretreated either with DMSO (DM, vehicle control), or the JNK inhibitors D-JNKI-1 (AM, 10 μM) or SP600125 (SP, 40 μM), then infected in the presence of drug for 60 minutes and finally in the presence of drug until the time of harvest, 8 hours post infection. Western blot for tubulin served as a loading control.

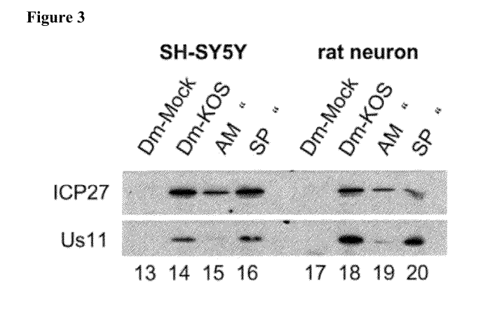

### DETAILED DESCRIPTION OF THE INVENTION

The present invention is based, in part, on the discovery that inhibition of c-Jun N-terminal kinase (JNK) in cells of the cochlea can be used to treat MD. Accordingly the present invention provides methods for treating MD by administering to a subject in need of such treatment a therapeutically effective amount of one or more JNK inhibitors.

According to the present invention, JNK is a member of the family of mitogen-activated protein kinases (MAPK), signal transmitting enzymes that respond to extracellular stimuli, e.g. stress or proinflammatory cytokines, and regulate various cellular activities, e.g. gene expression, differentiation, cell proliferation and differentiation, cytokine production and apoptosis (Garrington and Johnson, 1999; Manning and Davis, 2003). MAPK include extracellular signal-regulated kinases (ERK 1/2), p38 and c-Jun-N-terminal kinases (JNK 1/2/3) (Canlon et al., 2007).

JIP scaffold protein is a critical component of a MAPK signal transduction pathway that contributes to JNK activation in response to the exposure of cells to environmental stress (Wang et al., 2003). JNKs phosphorylate the N-terminal transactivation domain of the transcription factor c-Jun as well as other nuclear substrates including ATF-2 and Elk-1, and nonnuclear substrates such as Bc1-2 family members (Bogoyevitch et al., 2004). JIP retains JNK and other members of the MAPK-JNK signal cascade in the cytoplasm. A deficiency of activated JNK in the cell prevents c-Jun phosphorylation, thereby preventing the formation of transcription complexes. In turn, this prevents e.g. further progress along the JNK mediated apoptotic pathway or JNK mediated activation of genes which are encoding inflammatory molecules.

In general, inhibition of JNK prevents formation of transcription complexes and further progress along the apoptotic pathway or activation of genes which encode inflammatory molecules. According to the present invention, JNK inhibitor suitable for the methods of the present invention can be any biological or chemical entity that inhibits or reduces one or more activities of JNK.

In certain embodiments, the JNK inhibitor is a peptide. In some embodiments, peptide inhibitors of JNK useful for the methods of the invention comprises an amino acid sequence derived from the JNK binding domain of islet-brain 1 protein or islet-brain 2 protein, which are also known as JNK-interacting protein (JIP) 1 and 2, respectively. See Bonny et al., 2001, which is hereby incorporated by reference in its entirety. The JIP family of proteins has been shown to function as scaffold proteins in JNK signaling cascades. See Weston and Davis, Science 292: 2439-2440, 2001. For example, in such embodiments, the JNK peptide inhibitor comprises or consists of an amino acid sequence with substantial sequence homology to a sequence of DQSRPVQPFLNLTTPRKPR (SEQ ID NO: 1), RPKRPTTLNLFPQVPRSQD (SEQ ID NO: 4), DTYRPKRPTTLNLFPQVPRSQDT (SEQ ID NO: 13), TDQSRPVQPFLNLTTPRKPRYTD (SEQ ID NO: 15), HKHRPTTLRLTTLGAQDS (SEQ ID NO: 17), SDQAGLTTLRLTTPRHKH (SEQ ID NO: 19), RPKRPTTLNLF (SEQ ID NO: 21), or FLNLTTPRKPR (SEQ ID NO: 23). In other embodiments, the JNK peptide inhibitor comprises or consists of an amino acid sequence with substantial sequence homology to a sequence of RPKRPKTLNLF (SEQ ID NO: 25), FLNLTKPRKPR (SEQ ID NO: 27), RPKRPTFLNLF (SEQ ID NO: 29), FLNLFTPRKPR (SEQ ID NO: 31), RPKRPTSLNLF (SEQ ID NO: 33), FLNLSTPRKPR (SEQ ID NO: 35), RPKRPTTLNLD (SEQ ID NO: 37), DLNLTTPRKPR (SEQ ID NO: 39), PKRPTTLNLF (SEQ ID NO: 41), or FLNLTTPRKP (SEQ ID NO: 43). In some embodiments, the JNK peptide inhibitor comprises an amino acid sequence derived from the JNK binding domain of (JNK)-interacting protein-3 (JIP3) (Genbank Accession # NP_{—}055948.2). In certain embodiments, the JNK peptide inhibitor comprises or consists of a sequence selected from SEQ ID NO: 1, 4, 13, 15, 17, 19, 21, 23, 25, 27, 29, 31, 33, 35, 37, 39, 41, and 43.

The JNK inhibitor peptides can also be derived from c-Jun proteins. For example, a synthetic peptide comprising the JNK binding region on c-Jun, which corresponds to amino acids 33-79, is described in U.S. Pat. No. 6,514,745 as a competitive inhibitor of the naturally occurring c-Jun to decrease the amount of c-Jun activation by JNK. A cell-permeable 37-mer peptide consisting of the human c-Jun 6 domain (amino acids 33-57) sequence and the HIV-TAT protein transduction domain (amino acids 47-57), fused by a γ-aminobutyric acid (GABA) spacer (e.g., Ac-YGRKKRRQRRR-gaba-ILKQSMTLNLADPVGSLKPHLRAKN-NH2 (SEQ ID NO: 45)) was shown to specifically disrupt c-Jun/JNK complex formation and the subsequent phosphorylation and activation of c-Jun by JNK both in vitro and in intact cells. See Holzberg et al., J Biol Chem. 278(41):40213-23, 2003. Thus, in one embodiment, the JNK inhibitor used in the pharmaceutical compositions and methods of the invention comprises or consists of the SEQ ID NO: 45.

In some embodiments, the JNK inhibitor peptide binds to JNK. In other embodiments the JNK peptide inhibitor inhibits the activation of one or more components of the JNK signaling cascade, such as activation of a transcription factor, e.g. c-Jun, ATF-2, ELK-1, or p53. Other suitable JNK peptide inhibitors that may be used in the pharmaceutical compositions and methods of the invention are those described in U.S. Pat. Nos. 6,410,693; 6,610,820; 8,236,924; 8,080,517; and 8,183,339, each of which is hereby incorporated by reference in its entirety.

JNK peptide inhibitors comprising the amino acid sequences described herein may comprise about 15, about 20, about 25, about 30, about 35, about 40, about 45, about 50 or more amino acids. In some embodiments, the JNK peptide inhibitors comprise no more than 50 amino acids. In other embodiments, the JNK peptide inhibitors comprise no more than 35 amino acids. In certain embodiments, the JNK peptide inhibitors comprise from about 20 amino acids to about 50 amino acids or from about 25 amino acids to about 40 amino acids.

The JNK inhibitor peptides can be polymers of L-amino acids, D-amino acids, or a combination of both. For example, in some embodiments, the peptides are D retro-inverso peptides. The term “retro-inverso isomer” refers to an isomer of a linear peptide in which the direction of the sequence is reversed, the term “D-retro-inverso isomer” refers to an isomer of a linear peptide in which the direction of the sequence is reversed and the chirality of each amino acid residue is inverted. See, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the invention may be made into a D retro-inverso peptide by synthesizing a reverse of the sequence for the corresponding native L-amino acid sequence.

In some embodiments, the JNK peptide inhibitors comprise an amino acid sequence in which all of the chiral amino acids are in the D configuration. In other embodiments, the JNK peptide inhibitors comprise an amino acid sequence in which all of the chiral amino acids are in the L configuration. All amino acids except glycine can occur in two isomeric forms, because of the possibility of forming two different enantiomers around the central carbon atom. Thus, “chiral amino acids” refer to amino acids that have four different substituents attached to the central carbon atom.

The JNK peptide inhibitors that can be used in the pharmaceutical compositions and methods of the present invention further include derivatives, fragments, homologs, analogs and conservative variants of JNK inhibitor peptides herein described. As used herein, a conservative variant refers to an alteration in the amino acid sequence that does not adversely affect the biological functions of the peptide. A substitution, insertion or deletion is said to adversely affect the peptide when the altered sequence prevents or disrupts a biological function associated with the peptide. For example, the overall charge, structure or hydrophobic/hydrophilic properties of the peptide may be altered without adversely affecting a biological activity. Accordingly, the amino acid sequence can be altered, for example to render the peptide more hydrophobic or hydrophilic, without adversely affecting the biological activities of the peptide.

Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the invention are peptides having mutated sequences such that they remain homologous, e.g. in sequence and in function with a protein having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. In some embodiments, substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. In some embodiments, substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine.

Derivatives, fragments, and analogs of the peptide inhibitors described herein are defined as sequences of at least 4 contiguous amino acids, a length sufficient to allow for specific recognition of an epitope. The length of the fragments is less than the length of the corresponding full-length polypeptide from which the JNK inhibitor peptide is derived. Derivatives and analogs may be full length or other than full length, if the derivative or analog contains a modified nucleic acid or amino acid. Derivatives or analogs of the JNK inhibitor peptides include, e.g., molecules including regions that are substantially homologous to the peptides, in some embodiments, by at least about 30%, 50%, 70%, 80%, or 95%, 98%, or even 99%, identity over an amino acid sequence of identical size or when compared to an aligned sequence in which the alignment is done by a computer homology program known in the art. For example sequence identity can be measured using sequence analysis software (Sequence Analysis Software Package of the Genetics Computer Group, University of Wisconsin Biotechnology Center, 1710 University Avenue, Madison, Wis. 53705), with the default parameters therein. In one embodiment, the JNK peptide inhibitors comprise a sequence that is at least 80% identical to any one of SEQ ID NOs: 1 to 4 and 13-45. In another embodiment, the JNK peptide inhibitors comprise a sequence that is at least 90% identical to any one of SEQ ID NOs: 1 to 4 and 13-45. In still another embodiment, the JNK peptide inhibitors comprise a sequence that is at least 95% identical to any one of SEQ ID NOs: 1 to 4 and 13-45.

Where a particular polypeptide is said to have a specific percent identity to a reference polypeptide of a defined length, the percent identity is relative to the reference peptide. Thus, by way of example, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide, which is 50% identical to the reference polypeptide over its entire length. Of course, other polypeptides will meet the same criteria.

Another variation of the JNK peptide inhibitors is the linking of from one to fifteen amino acids or amino acid analogs to the N-terminal or C-terminal amino acid of the JNK peptide inhibitors described herein. Analogs of the JNK peptide inhibitors can be prepared by adding from one to fifteen additional amino acids to the N-terminal, C-terminal, or both N- and C-terminals, of an active peptide inhibitor, where such amino acid additions do not adversely affect the ability of the peptide to bind to JNK.

JNK-inhibitor peptides are obtained or produced by methods well-known in the art, e.g. chemical synthesis or genetic engineering methods. For example, a JNK peptide inhibitor, including a desired region or domain, may be synthesized by use of a peptide synthesizer. Alternatively, a JNK peptide inhibitor can be synthesized by recombinant expression by inserting a vector encoding the JNK peptide inhibitor into an appropriate host cell and culturing the host cell under conditions to promote expression. Suitable host cells include, but are not limited to, mammalian cells, insect cells, yeast cells, and bacteria cells. The JNK peptide inhibitors can also be synthesized using cell-free translation systems known in the art.

In certain embodiments, the JNK peptide inhibitors are chimeric peptides comprising a JNK binding domain fused to a protein transduction domain (PTD). PTDs are heterogeneous in size and lack sequence homology, although most share a positive charge and are amphipathic. In certain embodiments, PTDs can be antimicrobial peptides such as protegrin 1, Bactenecin 7, Buforin, and Maginin; a host of arginine-rich RNA- and DNA-binding peptides (e.g., HIV-1 transactivating protein (TAT) and Drosophila homeodomain transcription factor Antennapedia (a.k.a. Penetratin); chimeric PTDs such as Transportan; lysine- and arginine-rich peptides derived from phage-display libraries; polyarginine; and most recently, β-homolysine oligomers. See Fisher et al., 2001; Lindsay, 2002; Tung et al., 2002; Bogoyevitch et al., 2002; and Garcia-Echeverria et al., 2003, each of which is hereby incorporated by reference in its entirety. In certain embodiments, the PTDs are reverso-, retro-inverso, and enantio-forms of any of the PTDs described herein. Exemplary PTDs that may be fused to JNK-binding domains include PTDs derived from HIV TAT protein (e.g. GRKKRRQRRRPP (SEQ ID NO: 5) or PPRRRQRRKKRG (SEQ ID NO: 6)), Antennapedia protein (e.g. RQIKIWFQNRRMKWKK (SEQ ID NO: 7) or RRMKWKK (SEQ ID NO: 8)), SynB1 (e.g. RGGRLSYSRRRFSTSTGR (SEQ ID NO: 9)), SynB3 (RRLSYSRRRF (SEQ ID NO: 10)), SynBS (RGGRLAYLRRRWAVLGR (SEQ ID NO: 11)) or polyarginine (RRRRRRRR (SEQ ID NO: 12)). The PTD sequence can be fused to the N-terminus or C-terminus of the JNK-binding domain peptide. A linker of 1 to 10 amino acids can be inserted between the PTD sequence and the JNK-binding domain sequence. In some embodiments, a linker of two proline residues is used.

In particular embodiments, the PTD fused to the JNK-binding domain is derived from the TAT protein. In such embodiments, the chimeric peptides may comprise or consist of a sequence of:

(SEQ ID NO: 2)

DQSRPVQPFLNLTTPRKPRPPRRRQRRKKRG;

(SEQ ID NO: 3)

GRKKRRQRRRPPRPKRPTTLNLFPQVPRSQD;

(SEQ ID NO: 14)

GRKKRRQRRRPPDTYRPKRPTTLNLFPQVPRSQDT;

(SEQ ID NO: 16)

TDQSRPVQPFLNLTTPRKPRYTDPPRRRQRRKKRG;

(SEQ ID NO: 18)

GRKKRRQRRRPPHKHRPTTLRLTTLGAQDS;

(SEQ ID NO: 20)

SDQAGLTTLRLTTPRHKHPPRRRQRRKKRG;

(SEQ ID NO: 22)

GRKKRRQRRRPPRPKRPTTLNLF;

(SEQ ID NO: 24)

FLNLTTPRKPRPPRRRQRRKKRG;

(SEQ ID NO: 26)

GRKKRRQRRRPPRPKRPKTLNLF;

(SEQ ID NO: 28)

FLNLTKPRKPRPPRRRQRRKKRG;

(SEQ ID NO: 30)

GRKKRRQRRRPPRPKRPTFLNLF;

(SEQ ID NO: 32)

FLNLFTPRKPRPPRRRQRRKKRG;

(SEQ ID NO: 34)

GRKKRRQRRRPPRPKRPTSLNLF;

(SEQ ID NO: 36)

FLNLSTPRKPRPPRRRQRRKKRG;

(SEQ ID NO: 38)

GRKKRRQRRRPPRPKRPTTLNLD;

(SEQ ID NO: 40)

DLNLTTPRKPRPPRRRQRRKKRG;

(SEQ ID NO: 42)

GRKKRRQRRRPPPKRPTTLNLF;
or

(SEQ ID NO: 44)

FLNLTTPRKPPPRRRQRRKKRG.

The pharmaceutical compositions to be employed in the methods of the invention comprise a therapeutically effective amount of a JNK inhibitor, e.g., JNK peptide inhibitor or a pharmaceutically acceptable salt thereof and a pharmaceutically acceptable carrier or excipient. JNK peptide inhibitors included in the pharmaceutical compositions of the present invention can be in free form or the form of a salt, where the salt is pharmaceutically acceptable. Examples of such a pharmaceutically acceptable salt include, but are not limited to, those formed with organic acids (e.g. acetic, lactic, citric, malic, formaric, tartaric, stearic, ascorbic, succinic, benzoic, methanesulfonic, toluenesulfonic, or pamoic acid), inorganic acids (e.g., hydrochloridic, nitric, diphosphoric, sulphuric, or phosphoric acid), and polymeric acids (e.g., tannic acid, carboxymethyl cellulose, polylactic, polyglycolic, or copolymers of polylactic-glycolic acids). In one particular embodiment, the JNK peptide inhibitor is present in the pharmaceutical composition as an acetate salt.

Pharmaceutical compositions for any route of administration of this invention contain a therapeutically effective amount of the JNK peptide inhibitor, and, as may be necessary, inorganic or organic, solid or liquid pharmaceutically acceptable carriers or excipients. Pharmaceutical compositions suited for topical administration to the inner ear include aqueous solutions or suspensions, which, e.g. in the case of lyophilized formulations that contain the active ingredient alone or together with a carrier, may be prepared prior to use. They further include gels, which may be biodegradable or non-biodegradable, aqueous or non-aqueous, or microsphere based. Examples of gel-forming biocompatible polymers include, but are not limited to, hyaluronic acid resp. hyaluronates, lecithin gels, (poly)alanine derivatives, pluronics, poly(ethyleneglycol), poloxamers, chitosans, xyloglucans, collagens, fibrins, polyesters, poly(lactides), poly(glycolide) or their co-polymers PLGA, sucrose acetate isobutyrate, and glycerol monooleate. In some embodiments, gels are administered into the middle ear, release the peptide inhibitor over an extended period of time, and allow for a high percentage of the peptide inhibitor to be delivered into the inner ear.

Hyaluronic acid, which can be used as the biocompatible polymer in the pharmaceutical composition of the present invention, is a physiological substance that is widely distributed in the extracellular matrix of connective tissues in all organs of the body. It occurs in various molecular weights and is reported to be non-antigenic. Moreover, it has an excellent biocompatibility and is also biodegradable. Hyaluronic acid is a naturally occurring polysaccharide, a glycosaminoglycan composed of a long-chain polymer containing repeating disaccharide units of sodium glycuronate-N-acetylglucosamine. The main properties of hyaluronic acid are that it binds water and hence forms a degradable gel with high viscosity. The viscosity of the hyaluronic acid solutions increases with concentration and molecular weight. The JNK peptide inhibitors can be either dissolved or suspended in the hyaluronic acid gel. In some embodiments, the pharmaceutical compositions comprise about 0.5% to about 2% of hyaluronic acid. In other embodiments, the pharmaceutical compositions comprise about 0.7% to about 0.9% of hyaluronic acid.

The pharmaceutical compositions may be sterilized and/or may contain adjuvants, e.g. preservatives, stabilizers, wetting agents and/or emulsifiers, salts for regulating the osmotic pressure and/or buffers. In some embodiments, the pharmaceutical compositions comprise a buffer that buffers the pH of the composition from about 6.0 to about 7.4. In certain embodiments, the pharmaceutical compositions comprise a phosphate or citrate buffer. In related embodiments, the phosphate or citrate buffer buffers the pH of the composition to about 6.2.

The pharmaceutical compositions of the invention may, if desired, contain further pharmacologically active substances or other components, such as antibiotics, e.g., fluoroquinolones, anti-inflammatory agents, e.g., steroids, cortisone, analgesics, antipyrine, benzocaine, procaine, or antihistamines, e.g. betahistine or bilastine etc. The pharmaceutical compositions may be prepared by any of the methods well known in the art of pharmacy, e.g. by conventional mixing, granulating, confectioning, dissolving or lyophilizing methods, and contain from about 0.01 to 100%, preferably from about 0.1 to 50% (lyophilisates up to 100%), of active ingredient.

The pharmaceutical compositions comprising a JNK peptide inhibitor can be administered to a subject orally, intravenously, subcutaneously, intraperitoneally, intramuscularly, rectally or topically, such as intranasally. In certain embodiments, topical administration to the inner ear is used. In some embodiments, a therapeutically effective amount of a pharmaceutical composition comprising a JNK peptide inhibitor is able to reach cochlear cells.

Administration of the pharmaceutical composition to the inner ear may be accomplished by various delivery techniques. Such techniques include the use of devices or drug carriers to transport and/or deliver the JNK peptide inhibitor in a targeted fashion to the membranes of the round or oval window, where it diffuses into the inner ear or is actively infused. Examples are otowicks (see e.g. U.S. Pat. No. 6,120,484 to Silverstein, incorporated herein by reference), round window catheters (see e.g. U.S. Pat. Nos. 5,421,818; 5,474,529; 5,476,446; 6,045,528; all to Arenberg, or U.S. Pat. No. 6,377,849 and U.S. Patent Publication No. 2002/0082554 to Lenarz, all of which are incorporated herein by reference), microimplants (see e.g. WO2004/064912 by Jukarainen et al.) or various types of gels, foams, fibrins or other drug carriers, which are placed in the round window niche or on the oval window, and loaded with the compound for sustained release (see e.g. WO 97/38698 by Manning; Silverstein et al., Otolaryngology—Head and Neck Surgery 120 (5): 649-655 (1999); Balough et al., Otolaryngology—Head and Neck Surgery 119 (5): 427-431 (1998), each of which is hereby incorporated by reference in its entirety). Other suitable delivery techniques include the use of devices which are inserted into the cochlear duct or any other part of the cochlea (see e.g. U.S. Pat. No. 6,309,410 to Kuzma, incorporated herein by reference). The pharmaceutical composition comprising a JNK peptide inhibitor may also be administered to the inner ear by intratympanic injection, where the composition is injected into the middle ear over the area of the target inner-middle ear interface tissue structure, such as the round window niche (see e.g. Light J. and Silverstein H., Current Opinion in Otolaryngology & Head and Neck Surgery 12: 378-383 (2004)). The injection may be performed directly through the tympanic membrane (tympanopunction), through a myringotomy, through a ventilating tube inserted into the tympanic membrane, or through an opening of the tympanic membrane (e.g. by tympanomeatal flap). In some embodiments, the volume of the formulation to be injected is between about 50 and about 500 microlitres. In some embodiments, the volume of the formulation to be injected is between 150 and 250 microliters. In some embodiments, the method of administration to the inner ear is by diffusion across the round window membrane, which is relatively easily accessible from the middle ear space, and allows the inner ear to remain intact, thus avoiding any potential problems from leaking intracochlear fluids. Thus, in some embodiments, the pharmaceutical composition is delivered to the middle ear.

Pharmaceutical compositions which cannot be injected or infused by any of the aforementioned means may be deposited onto the target inner-middle ear interface structure across a small opening in the tympanic membrane with the aid of surgical instrument.

In some embodiments, the pharmaceutical composition comprising the peptide inhibitor of JNK is administered to the subject less than about four weeks, less than about three weeks, less than about two week, less than about one week, less than about six days, less than about five days, less than about four days, less than about three days, less than about two days, less than about one day, less than about 20 hours, less than about 15 hours, less than about 12 hours, less than about 8 hours, less than about 4 hours, less than about 2 hours, less than about 1 hour, less than half hour, less than 10 mins, less than 5 mins, or less than 1 min after the appearance of at least one, at least two, at least three or more symptoms of MD, or appearance of one or more prodromes of MD. In some embodiments, the pharmaceutical composition comprising the peptide inhibitor of JNK is administered to the subject within 48-72 hours after the appearance of one or more symptoms or prodromes of MD. In some embodiments, the pharmaceutical composition is administered to a subject suspected to be attacked by, or is being attacked by MD.

The pharmaceutical composition can be administered prior to, during or after an attack of MD, for example, prior to, during or after one or more symptoms of MD appear. The amount to be administered may vary, depending upon the method of administration, duration of therapy, the condition of the subject to be treated, the severity of the symptoms, the particular JNK peptide inhibitor used, and ultimately will be decided by the attending physician. In some embodiments, the pharmaceutical composition is administered to a patient through intratympanic injection, and the duration of the therapy may ranges between about several seconds or several minutes. In some embodiments, the pharmaceutical composition is administered to a patient through an implant formulation or sustained release formulation, and the duration of therapy may range between about several hours, about several days, weeks or months, or even years. In some embodiments, the treatment is extended up to chronic treatment. In some embodiments, in the case of therapies of long duration, repeat doses of the pharmaceutical composition may be administered.

A therapeutically effective amount or dose is defined as an amount or dose effective to suppress, reduce, stabilize, or delay the severity, duration, and/or frequency of one or more symptoms of MD in a treated individual. A therapeutically effective amount or dose is also the amount effective to prevent or delay the onset of one or more symptoms of MD. In one embodiment, a therapeutically effective amount or dose of a JNK peptide inhibitor is an amount or dose effective to reduce the perception of one or more MD symptoms by the afflicted individual following administration of the composition. In another embodiment, a therapeutically effective amount or dose of a JNK peptide inhibitor is an amount or dose effective to reduce the severity of one or more symptoms of MD following administration of the composition. In another embodiment, a therapeutically effective amount or dose of a JNK peptide inhibitor is an amount or dose effective to reduce the frequency of MD episodes or one or more symptoms of MD following administration of the composition. In still another embodiment, a therapeutically effective amount or dose of a JNK peptide inhibitor is an amount or dose effective to reduce the duration of MD episodes or one or more symptoms of MD following administration of the composition.

As stated above, a therapeutically effective amount or dose may vary, depending on the choice of specific JNK peptide inhibitor, the severity of the MD to be treated and on the method of its administration. For example, a lower dose of a JNK peptide inhibitor with a higher binding affinity for JNK may be more effective than a JNK peptide inhibitor that binds with a lower affinity. Additionally, a higher dose of an intravenously administered JNK peptide inhibitor would be required than that of the same pharmaceutical composition administered locally to the round window membrane or oval window of the ear. In some embodiments, when the pharmaceutical composition is administered intratympanically or through local use, the therapeutically effective amount or dose of JNK peptide inhibitor to be delivered may range from about 0.001 mg to about 5 mg, such as about 0.001 mg, about 0.002 mg, about 0.003 mg, about 0.004 mg, about 0.005 mg, about 0.006 mg, about 0.007 mg, about 0.008 mg, about 0.009 mg, about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3.0 mg or more. In some embodiments, the dose is about 0.001 mg to about 3 mg. In some embodiments, the dose is about 0.05 mg to about 0.5 mg. In some embodiments, when the pharmaceutical composition is administered systemically, a higher dose can be used accordingly, such as about 0.01 mg to about 100 mg, e.g., about 10 mg to 30 mg. In some embodiments, the inhibitor comprising or consisting of a sequence selected from SEQ ID NOs: 1-4 and 13-45. For JNK peptide inhibitor with shorter sequences, the dose will be reduced according to their molecular weight.

In some embodiments, the JNK peptide inhibitor in the composition has a concentration of about 1 μM to about 500 μM, such as about 0.1 μM, about 0.5 μM, about 1 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 110 μM, about 120 μM, about 130 μM, about 140 μM, about 150 μM, about 160 μM, about 170 μM, about 180 μM, about 190 μM, about 200 μM, about 210 μM, about 220 μM, about 230 μM, about 240 μM, about 250 μM, about 260 μM, about 270 μM, about 280 μM, about 290 μM, about 300 μM, about 310 μM, about 320 μM, about 330 μM, about 340 μM, about 350 μM, about 360 μM, about 370 μM, about 380 μM, about 390 μM, about 400 μM, about 410 μM, about 420 μM, about 430 μM, about 440 μM, about 450 μM, about 460 μM, about 470 μM, about 480 μM, about 490 μM, about 500 μM, or more. In some embodiments, about 5 μM, about 10 μM, about 15 μM, about 20 μM, about 50 μM, about 100 μM or more. In some embodiments, the concentration of the JNK peptide inhibitor is about 25 to 250 μM, which is equivalent to about 0.1 mg/ml to about 5 mg/ml.

In some embodiments, such JNK inhibitor is capable of reaching the apical region of the cochlea and acting therein in therapeutically effective concentrations. In some embodiments, the JNK inhibitor is reaching the apical region immediately upon administration. In some embodiments, the JNK inhibitor is reaching the apical region less than about 24 hours, less than about 12 hours, less than about 6 hours, less than about 3 hours, less than about 2 hours, less than about 1 hour, less than about 0.5 hour, less than about 0.4, less than about 0.3, less than about 0.2 or less than about 0.1 hour of administration.

In some embodiments, intratympanic administration of a composition of the present invention to patients is made within about 1 week, about 6 days, about 5 days, about 4 days, about 72 hours, about 60 hours, about 48 hours, about 36 hours, about 24 hours, about 12 hours, about 6 hours, about 1 hour or less from the onset of one or more symptoms of MD, or during the onset of one or more symptoms or prodromes of MD. In some embodiments, the pharmaceutical composition comprising the peptide inhibitor of JNK is administered to the subject within 48-72 hours after the appearance of one or more symptoms or prodromes of MD.

According to the present invention, JNK inhibitors can be used to treat Meniére's Disease (MD). As used herein, the terms “treating” and “treatment” refer to an approach for obtaining beneficial or desired results including clinical results, and may include even minimal changes or improvements in one or more measurable symptoms of the disease or condition being treated. A treatment is usually effective to reduce at least one symptom of a condition, disease, disorder, injury or damage. Exemplary measures of clinical improvement will be apparent to persons skilled in the art. Examples include, but are not limited to, one or more of the following: decreasing or completely resolving the severity, duration and/or frequency one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life, etc.

In general, MD is an idiopathic condition characterized by sudden attacks of symptoms including, but not limited to, vertigo, dizziness, nausea, vomiting, hearing loss, tinnitus, sensation of pressure or pain in the affected ear, etc. In some embodiments, one or more of these symptoms may last for about a couple of hours to 24 hours, and may subside gradually, e.g., vertigo, nausea, vomiting, etc. In some other embodiments, one or more of these symptoms may be persistent and/or progressive, e.g., hearing loss, tinnitus and a sensation of pressure or pain in affected ears. In some other embodiments, treatment of MD by the methods of the present invention including protection of sensory cells, supporting cells and neurons in the inner ear, e.g., affected hair cells in the cochlea or vestibule. In some other embodiments, treatment of MD by the methods of the present invention including attenuating one or more symptoms of MD, and/or long-term outcomes of MD, such as progressive sensorineural hearing loss. In still some other embodiments, treatment of MD by the methods of the present invention also includes anti-apoptotic and anti-inflammatory effects. In still yet some other embodiments, treatment of MD by the methods of the present invention includes attenuating replication of HSV and other types of viruses.

In certain embodiments, following administration, pharmaceutical compositions of the present invention are able to reach the apical part of the cochlea, which is most affected in Meniére's Disease, and is able to transfect sensory cells and other cell types there.

In certain other embodiments, the patient has “classic Meniére's” which is considered to have the following symptoms:
Attacks of rotational vertigo that can be severe, incapacitating, unpredictable, and last anywhere from minutes to hours, but generally no longer than 24 hours. For some, prolonged attacks can occur, lasting from several days to several weeks, often causing the sufferer to be severely incapacitated. Nausea, vomiting, and sweating sometimes accompany vertigo, but are symptoms of vertigo, and not of Meniére's.
Fluctuating, progressive, unilateral (in one ear) or bilateral (in both ears) hearing loss, usually in lower frequencies. For some, sounds can appear tinny or distorted, and patients can experience unusual sensitivity to noises. It can be temporary, albeit significant, hearing loss. Hearing may improve after an attack, but often becomes progressively worse.
Unilateral or bilateral tinnitus. This can be an increase in volume of tinnitus.
A sensation of fullness or pressure in one or both ears.

In some embodiments, Meniére's Disease is diagnosed and evaluated based on the guideline of guidelines of the American Academy of Otolaryngology (Committee on Hearing and Equilibrium, OTOLARYNGOL HEAD NECK SURG 1995; 113:181-5, incorporated by references in its entirety for all purposes).

In some embodiments, treatment of MD by the methods of the present invention includes treating MD patients with one or all of these symptoms at some point. In other embodiments, treatment of MD by the methods of the present invention includes treating at least two of the above symptoms, e.g., come on together in an episode. In some embodiments, at least three of the above symptoms come on together in an episode. In some embodiments, all four of the above symptoms come on together in an episode. In some embodiments, at least one symptom is progressive sensorineural hearing loss.

In some other embodiments, MD treated by the methods of the present invention may begin in one ear and become bilateral as the disorder progresses. Depending on their intensity, MD symptoms may be just a nuisance for patients or negatively affect their quality of life, making it impossible to perform normal activities of daily living. In some embodiments, pharmaceutical compositions of the present invention can attenuate the frequency, severity, and/or duration of one or more symptoms of MD.

In some embodiments, MD treated by the methods of the present invention includes an imbalance of inner ear fluid homeostasis, e.g., an increase in production or a decrease in reabsorption of inner ear fluid.

In some embodiments, MD treated by the methods of the present invention includes endolymphatic hydrops. For example, the MD is caused at least partially due to endolymphatic hydrops.

In some embodiments, MD treated by the methods of the present invention includes excess potassium-rich endolymphatic fluid leaking through Reissner's membrane into sodium-rich perilymphatic fluid and contamination afterwards.

In some embodiments, MD treated by the methods of the present invention includes a viral infection. In some embodiments, the viral infection is caused by herpes simplex virus HSV (e.g., HSV-1 or HSV-2), Epstein Barr Virus, cytomegalovirus, varicella zoster virus (VZV), influenza B, Coxsackie B5 virus, respiratory syncytial virus, or any combinations thereof. In some embodiments, patients with MD have an infection of HSV. In some embodiments, MD treated by methods of the present invention includes harboring latent HSV in vestibular nerves. In some embodiments, the HSV is triggered later in life and leads to MD, when the patient is subjected to a stressful incident, such as head trauma or infection. In some embodiments, MD treated by the methods of the present invention includes sensorineural hearing loss, e.g., provoked by migration of nucleic acid and viral protein toxins from the vestibulum into the apical part of the cochlea. In some embodiments, treatment of MD by the methods of the present invention includes attenuating replication of one or more viruses and therefore reducing one or more symptoms of MD.

In some embodiments, treatment of MD including treatment of patients either independently or also diagnosed to have HSV infection. For example, the patient has antibodies to HSV. Exemplary methods for detecting HSV antibodies are described in Arnold and Niedermeyer, 1997. In some embodiments, herpes simplex DNA can be found in the endolymphatic sacs of the patient. Exemplary methods for detecting herpes simplex DNA are described in Linthicum, 2001 and Vrabec, 2003. In some embodiments, herpes simplex DNA can be found in the vestibular ganglion of the patient. In some embodiments, the patient with MD has a significant loss of vestibular ganglion cells in both the endolymph hydropic and non-hydropic ears. Exemplary methods for detecting loss of vestibular ganglion cells are described in Gacek, 2009. In some embodiments, the patient with MD has viral particles enclosed in transport vesicles, which can be determined by transmission electron microscopy of vestibular ganglion cells as described in Gacek, 2009. Each of the references mentioned is herein incorporated by reference in its entirety. In some embodiments, HSV cannot be detected in the patient with MD.

In some embodiments, one or more viruses can be detected in a patient with MD. Such viruses include, but are not limited to, HSV, Epstein Barr Virus, cytomegalovirus, and/or varicella zoster virus (VZV), influenza B, Coxsackie B5 virus and respiratory syncytial virus. Other inner ear pathogens and exemplary methods for detecting these pathogens are described in Welling et al., 1997, Yazawa et al, 2003, Gartner et al., 2008, Pyykko and Zou, 2008, Beyea et al., 2012, Arbusow et al., 1999, each of which is herein incorporated by reference in its entirety. In some embodiments, pharmaceutical compositions of the present invention can be used to attenuate replication of one or more viruses thereof.

In some embodiments, the patient with MD contains one or more viruses that are different from HSV. In some embodiments, the patient with MD contains Epstein Barr Virus but not HSV (e.g., HSV-1 or HSV-2). In some embodiments, the patient with MD contains cytomegalovirus, but not HSV (e.g., HSV-1 or HSV-2). In some embodiments, the patient with MD contains VZV, but not HSV (e.g., HSV-1 or HSV-2).

In some embodiments, a patient with symptoms of MD has one or more vascular risk factors for cerebral ischemia, such as vascular disorders and/or chronic hypoxia. Such risk factors include, but are not limited to dyslipidemia, obesity, hypertension, age >55, smoking, atherosclerosis, diabetes, sleep apnea, history of myocardial infarction, history of stroke, and history of TIA (See Foster and Breeze, 2013, incorporated by reference in its entirety).

In some embodiments, MD treated by the methods of the present invention includes circulation problems in the ear, such as increased pressure of an abnormally large amount of endolymph in the inner ear and/or from the presence of potassium in an area of the inner ear which causes contamination.

In some embodiments, MD treated by the methods of the present invention includes breaks in the membrane separating enholymph from the other inner ear fluid, perilymph.

In some embodiments, patients with MD treated by the methods of the present invention are exposed to one or more triggers that can set off attacks, wherein the triggers include, but are not limited to, stress, overwork, fatigue, emotional distress, additional illnesses, pressure changes, certain foods, and too much salt in the diet.

In some embodiments, one or more symptoms of MD in a patient to be treated vary before, during, between, after attacks, and/or during the late-stage of MD. In some embodiments, one or more symptoms of MD in a patient to be treated stay constant or substantially constant before, during, between, after attacks, and/or during the late-stage of MD.

In some embodiments, oncoming attacks are preceded by an “aura”, or one or more warning symptoms, which include, but are not limited to, balance disturbance, dizziness, lightheadedness, headache, increased ear pressure, hearing loss or tinnitus increase, sound sensitivity, vague feeling of uneasiness. In some embodiments, a pharmaceutical composition of the present invention is administered to a patient with said one or more warning symptoms before onset of MD.

In some embodiments, during an attack of early-stage MD, the patient may have one or more main symptoms including but not limited to, spontaneous, violent vertigo, fluctuating hearing loss, ear fullness (aural fullness) and tinnitus. In addition to the main symptoms, the patient may have one or more additional symptoms including but not limited to, anxiety, fear, diarrhea, blurry vision or eye jerking, nausea and vomiting, cold sweat, palpitations or rapid pulse, and trembling. In some embodiments, a pharmaceutical composition of the present invention is administered to a patient with one or more such additional symptoms and reduces or suppresses one or more such symptoms.

In some embodiments, following an attack, a period of extreme fatigue or exhaustion may occur, promoting the need for hours of sleep. In some embodiments, a pharmaceutical composition of the present invention administered to a patient is able to reduce or suppress the extreme fatigue or exhaustion.

In some embodiments, a patient with MD to be treated is symptom-free during the periods between two attacks. In some embodiments, a patient with MD to be treated has one or more symptoms during the periods between two attacks, including but not limited to, anger, anxiety, fear, worry, appetite change, clumsiness, concentration difficulty, distractibility, tendency to grope for words, diarrhea, fatigue, malaise, sleepiness, headache, heavy head sensation, lightheadedness (faintness), loss of self-confidence and self-reliance, nausea, queasiness, motion sickness, neck ache or stiff neck, palpitations or rapid pulse, cold sweat, slurred speech, sound distortion and sensitivity, unsteadiness (sudden falls, staggering or stumbling, difficulty turning or walking in poorly lit areas, tendency to look down or to grope for stable handholds), vision difficulties (problems with blurring, bouncing, depth perception, glare intensification, focusing, watching movement; difficulty looking through lenses such as binoculars or cameras), and vomiting. In some embodiments, a pharmaceutical composition of the present invention administered to a patient is able to reduce or suppress one or more of these symptoms.

In some embodiments, a patient with MD to be treated is in late-stage MD. Hearing loss is more significant and is less likely to fluctuate. Tinnitus and/or aural fullness may be stronger and more constant. Attacks of vertigo may be replaced by more constant struggles with vision and balance, including difficulty walking in the dark and occasional sudden loss of balance. Sometimes, drop attacks of vestibular origin (Tumarkin's otolithic crisis) occur in this stage of Meniére's disease. Some of these late-stage symptoms can become more problematic in conditions of low lighting, or with fatigue, or when a person is exposed to visually stimulating situations. In some embodiments, a pharmaceutical composition of the present invention administered to a patient is able to reduce or suppress the symptoms in a patient in late-stage MD.

In some embodiments, each attack of MD lasts for about 5 seconds, about 10 seconds, about 30 seconds, about 1 min, about 5 mins, about 10 mins, about 15 mins, about 20 mins, about 30 mins, about 1 hour, about 2 hours, about 5 hours, about 12 hours, about 16 hours, about 20 hours, about 24 hours, or longer.

In some embodiments, attacks occur with a frequency of about twice a day, about every day, about every two days, about every three days, about every four days, about every five days, about every six days, about every week, about every 1.5 week, about every two weeks, about every three weeks, about every four weeks, about every 1.5 month, about every 2 months, about every 3 months, about every 4 months, about every 5 months, about every 6 months, about every 7 months, about every 8 months, about every 9 months, about every 10 months, about every 11 months, about every 12 months, about every 1.5 year, or more.

In some embodiments, the pharmaceutical compositions of the invention are administered to a subject as a prophylactic measure to reduce the development of one or more symptoms of MD. In some embodiments, a patient has one, two, three, or more symptoms of Meniére's Disease. In some embodiments, the symptoms include, but are not limited to, vertigo, dizziness, tinnitus, hearing loss and the sensation of pressure or pain in the affected ear. In some embodiments, the pharmaceutical composition is administered to a patient with one or more warning symptoms described herein. In some embodiments, the pharmaceutical composition is administered to a patient with one or more early-stage symptoms of MD described herein. In some embodiments, the pharmaceutical composition is administered to a patient with symptoms between attacks described herein. In some embodiments, the pharmaceutical composition is administered to a patient with one or more late-stage symptoms of MD described herein.

In some embodiments, the pharmaceutical composition is administered to a patient with endolymphatic hydrops, who has not developed symptoms, or has only minor symptoms of MD.

In some embodiments, the pharmaceutical composition is administered to a patient with one or more types of viral infections, who has not developed symptoms, or has only one or more minor symptoms of MD.

In some embodiments, the pharmaceutical composition is administered to a patient with one or more vascular risk factors for cerebral ischemia, who has not developed symptoms, or has only one or more minor symptoms of MD.

There are generally three types of vertigo. The first is known as objective and describes when the patient has the sensation that objects in the environment are moving. The second type of vertigo is known as subjective and refers to when the patient feels as if he or she is moving. The third type is known as pseudovertigo, an intensive sensation of rotation inside the patient's head. Vertigo is often associated with nausea and vomiting as well as a balance disorder, causing difficulties with standing or walking. Patients with vertigo typically present with mild to moderate imbalance, nausea, vomiting, hearing loss, tinnitus, fullness, and pain in the ear.

In some embodiments, the patient to be treated has one or more types of vertigo symptom, which can be ameliorated, treated, and/or prevented by a pharmaceutical composition of the present invention. In some embodiments, signs and symptoms of vertigo present as a persistent (insidious) onset, for example, lasting for longer than one day. In some embodiments, signs and symptoms of vertigo present as an episodic (sudden) onset, for example, the vertigo last less than about 5 seconds, less than about 10 seconds, less than about 30 seconds, less than about 1 min, less than about 2 mins, less than about 5 mins, less than about 10 mins, less than about 30 mins, less than about 1 hour, less than about 2 hours, less than about 4 hours, less than about 8 hours, less than about 12 hours, less than about 24 hours. In some embodiments, a pharmaceutical composition of the present invention can attenuate the degree, duration, and/or frequency of vertigo attacks.

Tests for vertigo are well known in the art. The tests can be either on vestibular system (balance) function, such as videonystagmography (VNG), electronystagmography (ENG), Dix-Hallpike maneuver, rotation tests, head-thrust test, caloric reflex test, computerized dynamic posturography (CDP), CT scans and MRIs, or on auditory system (hearing) function, such as pure tone audiometry, speech audiometry, acoustic reflex, electrocochleography (ECoG), otoacoustic emissions (OAE), and the auditory brainstem response test.

In some embodiments, the patients with MD to be treated have developed associated conditions, including endocrine abnormalities, electrolyte imbalance, autoimmune dysfunction, medications, infections (e.g. parasitic infections) or hyperlipidemia.

In some embodiments, the patients with MD to be treated have tinnitus, which can be ameliorated, treated, and/or prevented by a pharmaceutical composition of the present invention. The tinnitus to be ameliorated, treated, and/or prevented with the methods of the invention may be acute, subacute, or chronic. In some embodiments, tinnitus is constant or intermittent, buzzing, humming, ringing, hissing, whistling or roaring. It may occur in one or both ears, continuously or sporadically. Usually, tinnitus associated with MD is subjective. In some embodiments, a pharmaceutical composition of the present invention can improve one or more aspects of tinnitus experienced by the patient. For example, tinnitus in a patient would be considered to be ameliorated if the loudness of the tinnitus, frequency of the tinnitus, and/or duration of the tinnitus perceived by the patient is reduced or the tinnitus is completely resolved. In some embodiments, a pharmaceutical composition of the present invention can attenuate the degree, duration, and/or frequency of tinnitus, or suppress tinnitus. Methods of assessing the severity and presence of tinnitus in a patient are known to those of skill in the art and can include, but are not limited to, numerical rating scales or validated psychometric questionnaires, such as the Tinnitus Handicap Inventory, the Tinnitus Reaction Questionnaire, and the Tinnitus Functional Index. See, e.g., Figueiredo et al., 2009; Kamalski et al., 2010; and Meikle et al., 2011, each of which is herein incorporated by reference in its entirety.

In some embodiments, the patients with MD to be treated have labyrinthitis symptoms, which can be ameliorated, treated, and/or prevented by a pharmaceutical composition of the present invention. Labyrinthitis is an inflammation of the labyrinths of the ear which contain the vestibular system of the inner ear. Causes include bacterial, viral, and fungal infections. It may also be caused by a head injury or allergies. Symptoms of labyrinthitis include difficulty maintaining balance, dizziness, vertigo, tinnitus, and hearing loss. Recovery may take one to six weeks; however, chronic symptoms may be present for years.

It appears that all patients with the classical symptoms of MD have endolymphatic hydrops (EH), but the opposite is not true, as not all patients with EH have MD (Merchant et al., 2005). Endolymphatic hydrops refers to an increase in the hydraulic pressure within the endolymphatic system of the inner ear. Endolymphatic hydrops may be either primary or secondary. Primary idiopathic endolymphatic hydrops occurs for no known reason. Secondary endolymphatic hydrops appears to occur in response to an event or underlying condition. For example, it can follow head trauma or ear surgery, and it can occur with other inner ear disorders, allergies, or systemic disorders (such as diabetes or autoimmune disorders). In some embodiments, the patients with MD to be treated only have primary endolymphatic hydrops, which can be ameliorated, treated, and/or prevented by a pharmaceutical composition of the present invention. In some embodiments, the patients with MD to be treated also have secondary endolymphatic hydrops, which can be ameliorated, treated, and/or prevented by a pharmaceutical composition of the present invention.

Causes of EH include, but are not limited to, metabolic disturbances, hormonal imbalances, autoimmune disease, and viral, bacterial, or fungal infections. Symptoms include hearing loss, vertigo, tinnitus, dizziness, imbalance, and aural fullness. Nystagmus may also be present. There is no vestibular or auditory test that is diagnostic of endolymphatic hydrops. Diagnosis is clinical-based on the physician's observations and on the patient's history, symptoms, and symptom pattern. The clinical diagnosis may be strengthened by the results of certain tests. For example, certain abnormalities in electrocochleography (which tests the response of the eighth cranial nerve to clicks or tones presented to the ear) or audiometry (which tests hearing function) may support a hydrops diagnosis. More recently, gadolinium has been used to visualize endolymphatic hydrops in Meniére's Disease patients.

In some embodiments, the patients with MD to be treated have dizziness, which can be ameliorated, treated, and/or prevented by a pharmaceutical composition of the present invention. Dizziness is an impairment in spatial perception and stability, which may lead to a sense of spatial disorientation, motion of the environment, or lightheadedness.

The word dizziness can refer to vertigo, presyncope (lightheadness), disequilibrium, or a non-specific feeling such as giddiness or foolishness. Vertigo represents about 25% of cases of occurrences of dizziness. Disequilibrium is the sensation of being off balance, and is most often characterized by frequent falls in a specific direction. Lightheadedness is the sensation of dizziness and/or feeling that one may be about to faint, which can be transient, recurrent, or occasionally chronic. In some cases, the individual may feel as though his or her head is weightless.

In some embodiments, the patients with MD to be treated have hearing loss symptoms, which can be ameliorated, treated, and/or prevented by a pharmaceutical composition of the present invention. Hearing loss (hearing impairment, or deafness) symptoms include, but are not limited to, hypoacusis, i.e. the loss of hearing acuity, or hyperacusis, i.e. certain sounds that seem too loud; difficulty following conversations when two or more people are talking; difficulty hearing in noisy areas; hard to tell high-pitched sounds from one another; less trouble hearing men's voice than women's voices; problems hearing when there is background noise; and voice that sound mumbled or slurred.

The severity of a hearing impairment can be ranked according to the additional intensity above a nominal threshold that a sound must be before being detected by an individual. It is measured in decibels of hearing loss, or dB HL. In some embodiments, the Hearing loss symptoms in a patient to be treated by a pharmaceutical composition of the present invention are mild, moderate, moderately severe, severe or profound:

Mild: for adults, between about 26 and 40 dB HL;

Moderate: between about 41 and about 60 dB L;

Severe: between about 61 and 90 dB HL;

Profound: about 91 dB HL or greater;

Totally deaf: have no hearing at all.

In some embodiments, diagnostic hearing loss may take any of the following forms:
1. The average (arithmetic mean) of hearing thresholds at 0.25, 0.5, and 1 kHz is 15 dB or more higher than the average of 1, 2, and 3 kHz.
2. In unilateral cases, the average of threshold values at 0.5, 1, 2, and 3 kHz is 20 dB or more poorer in the ear in question than on the opposite side.
3. In bilateral cases, the average of threshold values at 0.5, 1, 2, and 3 kHz is greater than 25 dB in the studied ear.
4. In the judgment of the investigator, the patient's hearing loss meets reasonable audiometric criteria for hearing loss characteristic of Meniere's disease. The rationale for using this criterion should be stated and justified for each case.

Although hearing usually fluctuates early in Meniere's disease, fluctuation is not universally present and is not essential to the diagnosis, provided that hearing loss is documented at some time, as above. In some embodiments, the determination of hearing change can be based on the four-tone average (arithmetic mean) of thresholds at 0.5, 1, 2, and 3 kHz. A change of 10 dB or more or a change in word recognition score (speech discrimination) of 15 percentage points or more is considered clinically significant. In case the pure-tone average and word recognition scores change in opposite directions, the pure-tone average will determine the overall nature of the change for reporting purposes. The four-tone average of 0.5, 1, 2, and 3 kHz takes into account the importance of high frequencies in normal hearing. This particular four-tone average is consistent with the 1985 version of the guidelines and the Academy's formula for calculation of hearing handicap.

Pharmaceutical compositions of the present invention can effectively protect against hearing loss in MD patients. In some embodiments, a pharmaceutical composition of the present invention can attenuate the degree, duration, and/or frequency of hearing loss, such as attenuating progressive sensorineural hearing loss. In some embodiments, the hearing loss is sensorineural hearing loss. In some embodiments, the hearing loss is progressive sensorineural hearing loss. Sensorineural hearing loss (SNHL) is a type of hearing loss in which the root cause lies in the vestibulocochlear nerve (cranial nerve VIII), the inner ear, or central processing centers of the brain. In MD patients, sensorineural hearing loss originates in inner ear. Sensorineural hearing loss can be mild, moderate, severe or profound, including total deafness. In some embodiments, during one episode of attack, the hearing loss occurs in one ear. In some embodiments, during one episode of attack, the hearing loss occurs in both ears, either simultaneously, or consequently. In MD patients, the hearing loss usually affects one ear, which typically loses sensitivity to low-frequency (bass) sounds the most. As well as being harder to hear, sounds may appear “tinny” or distorted. Loud sounds may cause more discomfort than normal (loudness intolerance). The hearing loss fluctuates over time but eventually affects all sound frequencies. Complete hearing loss (dead ear) is uncommon. However, the hearing loss typically worsens to a moderate to severe loss.

Progressive hearing loss is different from acute hearing loss. For acute sensorineural hearing loss, a patient may lose hearing of at least 30 dB, at least 40 dB, at least 50 dB, at least 60 dB, at least 70 dB or more within a short period of time due to a single, non-recurring insult to the inner ear. For example, the hearing loss happens within less than 72 hours, less than 60 hours, less than 48 hours, less than 36 hours, less than 24 hours, less than 12 hours, less than 8 hours, less than 4 hours, less than 2 hours after onset of an attack. In some embodiments, the acute hearing loss is sudden hearing loss, which is defined as greater than 30 dB hearing reduction, over at least three contiguous frequencies, occurring over a period of 72 hours or less. Depending, inter alia, on the severity of the acute hearing loss, all or part of it may recover thanks to physiological repair mechanisms within the cochlea. Part of the acute sensorineural hearing loss may persist, however. In MD attacks, hearing loss is also acute in each episode and typically recovers, at least partly. However, due to the recurrent nature of MD, hearing loss accumulates and gradually worsens as MD progresses. For example, depending on the frequency and severity of MD attacks, a patient may lose no more than 5 dB, no more than 10 dB, no more than 15 dB, no more than 20 dB, no more than 25 dB, no more than 30 dB, over at least one week, at least two weeks, at least three weeks, at least one month, at least two months, at least three months, at least four months, at least five months, at least six months, at least one year, at least two years, at least 5 years, or more. In some embodiments, the symptom of progressive hearing loss is continuously worsened. In some embodiments, the symptom of the progressive hearing loss may have temporary recovery, but the general trend is getting worse as MD progresses.

In some embodiments, the patients with MD to be treated have pressure or pain in the affected ear, which can be ameliorated, treated, and/or prevented by a pharmaceutical composition of the present invention. The pressure or pain can be aural fullness or pressure, a sensation of plugging, clogging or congesting in the ear that worsens when an attack happens. In some embodiments, yawning, swallowing, or other usual methods for eliminating this sensation have failed to work. In some embodiments, the ear fullness may accompany other symptoms, including but not limited to, pain or tenderness around the ear or in the bone behind the ear, pruritus (itching), pus or other secretions draining from the ear, redness, warmth or swelling.

In some embodiments, the patients with MD to be treated are children. In some embodiments, the patients with MD to be treated are adults. In some embodiments, the patients are at least 40 years old, at least 50 years old, at least 60 years old, or more.

In some embodiments, pharmaceutical compositions of the present invention can ameliorated, treated, and/or prevented one or more symptoms of MD in a clinically relevant, statistically significant and/or persistent fashion. In some embodiments, administration of a pharmaceutical composition of the present invention provides statistically significant therapeutic effect for ameliorating, treating, and/or preventing one or more symptoms of MD. In one embodiment, the statistically significant therapeutic effect is determined based on one or more standards or criteria provided by one or more regulatory agencies in the United States, e.g., FDA or other countries. In some embodiments, the statistically significant therapeutic effect is determined based on results obtained from regulatory agency approved clinical trial set up and/or procedure. In some embodiments, the statistically significant therapeutic effect is determined based on the criteria of guidelines of the American Academy of Otolaryngology (Committee on Hearing and Equilibrium, OTOLARYNGOL HEAD NECK SURG 1995; 113:181-5, incorporated by references in its entirety for all purposes).

In some embodiments, the statistically significant therapeutic effect is determined based on a patient population of at least 50, 100, 200, 300, 400, 500, 600, 700, 800, 900, 1000, or more. In some embodiments, the statistically significant therapeutic effect is determined based on data obtained from randomized and double blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%. In some embodiments, the statistically significant therapeutic effect is determined on approval of Phase III clinical trial of the methods provided by the present invention, e.g., by FDA in the US.

In some embodiment, the statistically significant therapeutic effect is determined by a randomized double blind clinical trial of a patient population of at least 50, 100, 200, 300 or 350; treated with a pharmaceutical composition of the present invention, but not in combination with any other agent for treating MD symptoms. In some embodiment, the statistically significant therapeutic effect is determined by a randomized clinical trial of a patient population of at least 50, 100, 200, 300 or 350 and using any commonly accepted criteria for MD symptoms assessment, such as the criteria described herein.

In general, statistical analysis can include any suitable method permitted by a regulatory agency, e.g., FDA in the US or China or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

In some embodiments, a composition of the present invention can be combined with one or more additional active agents to treat MD. As used herein, the term “combined” or “combination” refers to a treatment in which compositions of the present invention and one or more additional active agents are used together, consequently, or intermediately. In some embodiments, at least one additional active agent is an antiviral agent. In some embodiments, the antiviral agent is acyclovir, famciclovir or ganciclovir. In some embodiments, at least one additional active agent is a drug for reducing vascular risk factors. In some embodiments, at least one additional active agent is for relieving vertigo, such as frequently starts with a low-salt diet and/or administration of diuretics to keep sodium concentrations in the inner ear from fluctuating and to reduce inner ear fluid volume. In some embodiments, the additional active agent contains anticholinergics (e.g. scopolamine), antihistamines (e.g. meclizine, dimenhydrinate, or promethazine), antidopaminergics (e.g. prochlorperazine or chlorpromazine), and/or monaminergics (e.g. amphetamines or ephedrine). In some embodiments, the additional active agent contains benzodiazepine diazepam, which acts as a vestibular suppressant through the GABAergic system and reduces the anxiety and panic that may occur with vertigo. In some embodiments, at least one additional active agent is a calcium channel blocker (e.g. cinnarizine), or a corticosteroid (e.g., dexamethasone, predinsolone), or vasodilators.

In some embodiments, a composition of the present invention is combined with a prostanoid FP receptor agonist such as the glaucoma drug latanoprost, or those described in US Patent Application 2005/0171054, which is incorporated by reference herein.

In some embodiments, a composition of the present invention is combined with an active agent that can slowly activate delayed rectifier potassium current, such as those described in U.S. Pat. No. 5,817,658, which is incorporated by reference herein. In some embodiments, the additional active agent is a benzodiazepine or derivatives thereof.

In some embodiments, a composition of the present invention is combined with an AMPA receptor antagonist, such as those described in US Patent Application 2006/0166969, which is incorporated by reference herein. In some embodiments, the additional active agent is a benzodiazepine.

In some embodiments, a composition of the present invention is combined with an additional active agent for treating dizziness and/or vertigo.

In some embodiments, a composition of the present invention is combined with an additional active agent for reducing endolymphatic pressure, such as those described in U.S. Pat. No. 6,245,820, which is incorporated by reference herein. In some embodiments, the additional active agent is a polyol. In some embodiments, the polyol is erythritol, maltitol, mannitol, xylitol, or isosorbitol.

In some embodiments, a composition of the present invention is combined with a treatment for MD consisting of an individualized water intake regime and administration of isosorbide with the aim of reducing EH, such as those described in US Patent Application 2006/0003016, which is incorporated by reference herein.

In some embodiments, a composition of the present invention is combined with an additional active agent for treating inner ear disorders. In some embodiments, the additional active agent is a corticosteroid. In some embodiments, the corticosteroid is a multiparticulate anti-inflammatory corticosteroid, such as those described in U.S. Pat. Nos. 8,030,297, 8,546,363 and 8,658,626, each of which is incorporated by reference herein. In some embodiments, the corticosteroid is dexamethasone. In some embodiments, the corticosteroid is formulated in a poloxamer gel for intratympanic injection.

In some embodiments, a composition of the present invention is combined with a vasopressin (VP) antagonist, such as OPC-31260 (a competitive antagonist of V2-R), WAY-140288, CL-385004, tolvaptan, conivaptan, SR 121463A and VPA 985. (Sanghi et al. Eur. Heart J. (2005) 26:538-543; Palm et al. Nephrol. Dial Transplant (1999) 14:2559-2562), each of which is incorporated by reference herein.

In some embodiments, a composition of the present invention is combined with treatments that are aimed at dealing with the immediate symptoms and prevention of recurrence, such as low-sodium diets, avoidance of caffeine, alcohol, and tobacco. In some embodiments, a composition of the present invention is combined with antihistamines (including meclizine (marketed under the names Antivert®, Bonine®, Dramamine®, Driminate) and other antihistamines), and central nervous system agents, including barbiturates and/or benzodiazepines, including lorazepam or diazepam. Other examples of drugs that are useful in relieving symptoms include muscarinic antagonists, including scopolamine. Nausea and vomiting are relieved by suppositories containing antipsychotic agents, including the phenothiazine agent prochlorperazine (marketed under the names Compazine®, Buccastem, Stemetil and Phenotil).

In some embodiments, a composition of the present invention is combined with an active agent that can reduce tinnitus symptoms, such as lidocaine, ketamine or esketamine, AM-101 (e.g., those disclosed in U.S. Pat. Nos. 8,268,866, 8,507,525, and U.S. Patent Application Publication Nos. 2005214338, 2006063802, 2010254907, and 20140017172, each of which is incorporated by reference herein), and selective neurotransmitter reuptake inhibitors, such as nortriptyline, sertraline, paroxetine, and benzodiazepines.

In some embodiments, a composition of the present invention is combined with an active agent for treating labyrinthitis. There are several treatments for labyrinthitis. Prochlorperazine is often prescribed as an antiemetic. Serotonin-reuptake inhibitors have been shown to stimulate new neural growth within the inner ear. Additionally, treatment with antibiotics is prescribed if the cause is a bacterial infection, and treatment with corticosteroids and antivirals is recommended if the condition is caused by a viral infection.

In some embodiments, a composition of the present invention is combined with an active agent for treating EH. Active agents for treating EH includes, but are not limited to, benzodiazepine, diuretics (to decrease the fluid pressure), corticosteroids, anti-bacterial, anti-viral, and anti-fungal agents.

In some embodiments, a composition of the present invention is combined with an active agent for treating EH.

### EXAMPLES

### Example 1

In the absence of a relevant and reliable animal model, the intracellular JNK inhibitor D-JNKI-1 (SEQ ID NO: 2) has not been tested for the treatment of viral infection in the inner ear so far. Modulation of JNK and/or p38 MAPK pathways is required for infection and replication of HSV-1, Epstein-Barr virus or VZV (Wei et al., 2009). Beckham et al., 2007 showed that reovirus infection results in activation of JNK and caspase-3 in the central nervous system (CNS). Treatment of reovirus-infected mice with D-JNKI-1 resulted in significantly prolonged survival of intracerebrally infected mice following an otherwise lethal challenge with T3D (100×50% lethal dose). Protection correlated with reduced CNS injury, reduced neuronal apoptosis, and reduced c-Jun activation without altering the viral titer or viral antigen distribution. As we demonstrate in cell assays below, application of D-JNKI-1 and to a lesser extent also the small molecule JNK inhibitor SP600125 reduced HSV yield and viral replication.

A single cycle herpes simplex virus infection assay was performed to determine the effect of JNK inhibitors on virus yield.

### Materials and Methods

To prepare a 1 mM stock solution, 5.15 mg of D-JNKI-1 acetate was suspended in 1 ml of water, vortexed for 2 minutes, and sterilized by passage through a PES filter; 50 μL aliquots were prepared and stored at −20° C.

Human neuroblastoma cells (SH-SY5Y) were maintained in DMEM plus 10% fetal calf serum, 1 mM HEPES (Gibco) and 2 μg/ml gentamicin (Gibco). For differentiation, monolayers of cells were rinsed in PBS and then maintained in Neurobasal medium (Gibco) plus 2% B27 supplement and 9% all-trans retinoic acid (ATRA).
Primary fetal rat cortex cells were maintained in Neurobasal medium plus 2% B27, 2 mM glutamine and 50 mg/ml gentamicin.

Monolayers of SH-SY5Y human neuroblastoma cells differentiated by treatment with ATRA, or fetal rat cortex neurons were pre-treated with 10-20 μM D-JNKI-1, 40 μM SP600125 (Calbiochem), or DMSO (D2650, Sigma-Aldrich) vehicle control for 30 minutes prior to infection. Monolayers were infected with the KOS strain of HSV-1 at a multiplicity of infection (MOI) of 5 for 60 minutes under conditions of pre-treatment. Following the inoculation period, monolayers were incubated under pre-treatment conditions and harvested at 20 hours postinfection.

Cells and media were collected, and subjected to three rounds of freezing and thawing. Serial ten-fold dilutions were prepared and aliquots seeded in triplicate onto monolayers of Vero cells. Following a 60-minutes infection period, the inoculum was removed and replaced with medium containing 0.3% methyl cellulose. After 3 days, the overlay was removed and the cell monolayer stained with 0.8% crystal violet in 50% ethanol and plaques counted to determine virus yield. Results were presented as % of yield relative to control conditions (DMSO vehicle treatment).

### Results

In differentiated human neuroblastoma cells and rat cortex neurons, treatment with either D-JNKI-1 or SP100625 resulted in substantially reduced virus yield. Triplicate values were all within <±0.5 SD.

### Differentiated Human Neuroblastoma Cells

| | | | |
|---|---|---|---|
| | D-JNKI-1 | D-JNKI-1 | SP600125 |
| DMSO | 10 μM | 20 μM | 40 μM |

First experiment

| | | | | |
|---|---|---|---|---|
| Pfu/culture | 3.5 × 10⁵ | 1.27 × 10⁵ | | 2.57 × 10⁵ |
| % of control | 100 | 37 | — | 72 |

Second experiment

| | | | | |
|---|---|---|---|---|
| Pfu/culture | 8.5 × 10⁵ | 2.23 × 10⁵ | 2.06 × 10⁵ | 1.76 × 10⁵ |
| % of control | 100 | 26 | 24 | 20 |

### Fetal Rat Neurons

| | | D-JNKI-1 | SP100625 |
|---|---|---|---|
| | DMSO | 10 μM | 40 μM |
| Pfu/culture | 1.33 × 10⁶ | 1.67 × 10⁵ | 8.0 × 10⁵ |
| % of control | 100 | 12 | 60 |

### Example 2

The ability of JNK inhibitors to affect accumulation of viral proteins representative of the immediate-early (IE), early (E) or late (L) kinetic classes was evaluated by a Western blot assay.

### Materials and Methods

Unless indicated otherwise, the same materials and methods were applied as for the experiment described under Example 1 and as laid out by Hargett et al., 2005.

Replicate monolayers of rat fetal cortex neurons or ATRA-differentiated SH-SY5Y cells were pre-treated with DMSO, D-JNKI-1 (10 μM) or SP100625 (40 μM) for 30 minutes, and then infected with wild type HSV-1 (KOS) or ICP27 null mutant d27 at a multiplicity of infection (MOI) of 5, or mock-infected under pre-treatment conditions for 60 minutes. The inoculum was removed and incubation continued under the pretreatment conditions until the time of harvest. Whole cell lysates were prepared at 8 h post infection. Aliquots of lysates were separated on 12% gels by standard polyacrylamide gel electrophoresis (SDS-PAGE) procedures. Proteins were transferred to polyvinylidene difluoride (PVDF) membranes, blocked in tris buffered saline with Tween 20 (TBST) with 5% milk and probed with monoclonal antibodies to ICP27 and Us11 and polyclonal antibodies to ICP8 and gC, followed by goat anti-rabbit and anti-mouse immunoglobulin secondary antibodies (Amersham Biosciences). Following incubation in luminol reagent, signals were captured on X-ray film. Western blot for α-tubulin served as a loading control (B-5-1-2 monoclonal antibody, Sigma).

### Results

In both SH-SY5Y cells and fetal cortex neurons, treatment with D-JNKI-1 resulted in moderate reductions in the accumulation of IE (ICP27) and E (ICP8) proteins, and severe reduction in L (gC and Us11) protein accumulation. Although SP600125 also showed a reduction, D-JNKI-1 had more profound effects on viral protein accumulation. These results were thus consistent with the virus yield assay performed under Example 1 that indicated reductions in virus replication in the presence of both JNK inhibitors.

### Example 3

An in vivo study with fluorescence labeled D-JNKI-1 was conducted to evaluate whether the compound could reach the cochlear apex and the vestibular system following a single dose round window membrane application.

### Materials and Methods

FITC-labeled D-JNKI-1 (NeoMPS, Strasbourg, France) was prepared in an isotonic NaCl solution at a concentration of 0.1 mM. Two groups of 3 adult chinchillas each were anesthetized (ketamine 40-60 mg/kg plus acepromazine 1-2 mg/kg; supplementary half doses as needed), and the middle ear space was opened using an inferior-posterior auricular approach to expose the round window membrane. A 100 μL syringe attached to a 30 gauge needle was used to deliver 30 μL of the test solution to the round window membrane. After applying the test solution, the head of the animal was maintained in a stable orientation for 30-40 minutes so that the solution remained on the round window.

Animals were sacrificed 1 or 3 hours after administration of the test solution. They were anesthetized and then killed by decapitation. The temporal bone was quickly removed, and fixed with 10% formalin in PBS. The whole cochlear basilar membrane and maculae of the utricle was micro-dissected out, and stained with TRIC-conjugated phalloidin (1:100) for 20 min. The cochlear basilar membrane and maculae of the utricle were mounted in glycerin on glass slides as a flat surface preparation, and then cover slipped.

Specimens were examined on a Carl Zeiss Laser Scanning Systems LSM 510. Images were captured and analyzed with Zeiss LSM Image Examiner (version 4,0,0,91, Carl Zeiss GmbH Jena). For a qualitative ranking of the FITC labeling intensity, all the samples were evaluated with the same microscope settings (400× magnification, pinhole: 108, detector gain: 692) and the same observer ranked the intensity of FITC labeling of the inner ear sensory epithelia. The rating was performed on a 5 point scale using the following criteria: 0=no labeling, 1=weak labeling, 2=moderate labeling, 3=strong labeling, 4=heavy labeling. Location along the length of the basilar membrane was determined by a calibrated ruler in the eyepiece of the microscope. The cochlear locations examined were 2 mm (hook region), 8 mm (upper base), 12 mm (middle turn), and 16 mm (apex) from the base of the cochlea. FITC labeling intensity was also examined in the middle of the maculae of the utricle using the same rating scale. The intensity of the laser was fixed.

### Results

FITC labeling was present in all regions of the cochlea 1 and 3 h post-treatment. Mean labeling intensity was highest near the base of the cochlea and lower the greater the distance from the base. However, strong labeling at the apex was observed already 1 hour after administration, and labeling was also observed in the utricle 1 h and 3 h post-treatment, although weaker.

| | Cochlea | | | | |
|---|---|---|---|---|---|
| | Lower | Upper | Middle | | |
| Fluores- | basal | basal | basal | | Vestibulum |
| cence | turn | turn | turn | Apex | Utricle |
| rating | (2 mm) | (8 mm) | (12 mm) | (16 mm) | (centre) |
| 1 hour | 4.0 (0.0) | 3.7 (0.6) | 3.3 (0.6) | 3.0 (0.0) | 0.7 (0.6) |
| 3 hours | 4.0 (0.0) | 3.7 (0.6) | 3.0 (1.0) | 2.7 (0.6) | 1.0 (0.0) |

### Example 4

### Efficacy of a Peptide Inhibitor of JNK in the Treatment of Inner Ear Tinnitus

c-Jun N-terminal kinase (JNK) is involved in apoptosis of stress injured hair cells and spiral ganglia neurons (Zine et al., 2004; Abi-Hachem et al., 2010), the principal mechanism of cell death in the cochlea following traumatic injury (Hu et al., 2002) or cochlear inflammation (Ma et al., 2000; Barkdull et al., 2007). AM-111 is a 31-amino acid cell-permeable peptide (SEQ ID NO: 2 in which all chiral amino acids are in the D configuration and the peptide is synthesized in the reverse order), formulated in a biocompatible hyaluronic acid gel. The chimeric peptide contains an effector domain derived from the scaffold protein islet-brain 1 which retains JNK in the cytoplasm fused to the Trans-Activator of Transcription (TAT) protein transduction domain (Bonny et al., 2001). Treatment with AM-111 was shown to be otoprotective in various models of cochlear insult: acute noise trauma (Wang et al., 2003; Wang et al. 2007; and Coleman et al. 2007), acute labyrinthitis (Barkdull et al., 2007), aminoglycoside ototoxicity (Wang et al., 2003), bacterial infection (Grindal et al., 2010), cochlear ischemia (Omotehara et al., 2011) and cochlear implantation trauma (Eshraghi et al., 2013). The breadth of AM-111's therapeutic spectrum suggests a key role of JNK in acute sensorineural hearing loss (ASNHL). However, no statistically significant effect of AM-111 on tinnitus has been previously reported.

A double-blind, randomized, placebo-controlled phase II study was conducted to evaluate the efficacy and safety of AM-111 in treating ASNHL and associated tinnitus. Eligible participants were aged 18 to 60 years and suffered from ASNHL (unilateral idiopathic sudden sensorineural hearing loss (ISSNHL), uni- or bilateral acute acoustic trauma (AAT)) with hearing loss of at least 30 dB and onset not more than 48 hours previously. The hearing loss was determined against a reference value: mean hearing threshold at the 3 most affected contiguous test frequencies (pure tone average, PTA) less corresponding mean hearing threshold of the contralateral ear (Plontke et al., 2007). In case of previously asymmetric hearing or bilateral ASNHL, thresholds from a previous audiogram or ISO-7029; 2000 norm values served as reference. The PTA frequencies determined at baseline remained fixed for all evaluations.

Exclusion criteria included history of Meniére's disease, autoimmune or radiation induced hearing 80 loss, endolymphatic hydrops or fluctuating hearing loss, suspected perilymph fistula, membrane rupture or retrocochlear lesion, barotrauma, air-bone gap >20 dB in 3 contiguous frequencies, previous ASNHL incident within the past 6 weeks, and acute or chronic otitis media or otitis externa. Women who were breast feeding, pregnant or who planned a pregnancy during the study, or women of childbearing potential who declared being unwilling or unable to practice an effective method of 85 contraception were excluded. Written informed consent was obtained from each patient prior to the performance of any study-specific procedures.

At baseline (Day 0), study participants were randomized to receive AM-111 (0.4 or 2.0 mg/mL) or placebo at a 2:1 ratio. The study consisted of a baseline assessment and 4 follow-up visits on Days 3, 7, 30, and 90. Baseline assessments included a general physical examination, vital signs, and a urine pregnancy test for women of childbearing age. At each study visit pure tone hearing thresholds, speech discrimination at 60 and 80 dB, and subjective tinnitus loudness were determined. Those patients reporting tinnitus were asked to rate its loudness “right now” on a numerical scale ranging from 0 (no tinnitus) to 10 (extremely loud).

Approximately 0.25 mL of the study drug was administered on Day 0 by intratympanic (i.t.) injection under local anesthesia through a small myringotomy with the patient's head placed in a position tilted 45° towards the unaffected ear. Patients remained in their reclined or supine position for approximately 30 minutes to allow for diffusion of the active substance into the cochlea. In case of bilateral AAT, only the worst affected ear was treated. Subjects whose PTA recovered <10 dB from baseline to Day 7 were given the option to receive oral prednisolone 50 mg (Ratiopharm, Ulm, Germany) b.i.d. for 5 days. Previous reports showed no difference in outcomes for corticosteroid therapy initiated within the first 24 hours or within the first week (Huy et al., 2005).

The sample size was determined based on an expected effect size of 0.6, a two-sided type I error rate of 5% and a statistical power of 80%. This resulted in a planned sample size of 102 patients per cohort (68 AM-111, 34 placebo), for a total of 204 patients.

Efficacy analyses were primarily performed on a modified “Intention to treat” (mITT) analysis set (treated patients with PTA measured on Day 3±1 or Day 7+4 days at most) and secondarily on the “Per Protocol” (PP) analysis set. The “Safety Population” analysis set included all patients who received an injection of the study medication. Random imputation was performed for missing PTA values at Days 7 and 30, and missing speech discrimination score (SDS) values at Day 7 based on the preceding value and the mean change observed in the respective treatment group (mITT set).

For continuous efficacy endpoints, analysis of covariance (ANCOVA) models were used including treatment group and initial frequency range as class effects, and baseline values of the respective endpoint as covariate. For the complete recovery rate, a logistic regression model was applied including treatment, initial frequency range, and baseline hearing loss as predictor variables. Initial frequency range was included in the models since spontaneous recovery has been reported to be more pronounced in the lower frequencies (Huy et al., 2005). The percentage of subjects with post-treatment remission of ASNHL-related tinnitus was compared using the Fisher exact test.

### Results

A total of 210 patients were screened for and enrolled into the AM-111 phase IIb study in 2 cohorts. Each cohort comprised 105 patients: 70 allocated to AM-111 high dose (2.0 mg/mL) together with 35 allocated to placebo, 68 allocated to AM-111 low dose (0.4 mg/mL) group together with 37 allocated to placebo. All patients received one i.t. injection of study drug and constituted the “Safety Population” analysis set (210 patients). 11 patients (5%) were lost to follow-up and 6 (3%) withdrew consent. A total of 197 patients were included in the modified “Intention to treat” analysis set. The most common reasons for exclusion were study visits not performed within stipulated schedule (5 patients) and taking prohibited medication (5 patients). A total of 188 patients were included in the per protocol analysis set; the most frequent reason for exclusion was violation of the 30 dB minimum hearing loss criterion (8 patients).

The majority of patients were male (61%), suffered from ISSNHL (92%) and had tinnitus as comorbidity (80%). On average, patients were treated 29 hours post ASNHL onset. The mean hearing loss at the 3 most affected test frequencies was 52.2 dB; the mean SDS was 52.3% (60 dB) and 67.6% (80 dB). Clinically relevant spontaneous nystagmus (defined as >5 beats/30 sec) was observed in 7% of patients. Overall, baseline characteristics were similar for treatment groups.

A total of 167 subjects had ASNHL-related tinnitus at baseline (52 subjects who received placebo and 115 subjects who received AM-111). Baseline incidence was 73.2%, 82.4%, and 84.3% in the placebo, AM-111 0.4 mg/mL and AM-111 2.0 mg/mL groups. See table below.

Tinnitus at Baseline: Frequencies of Onset with Respect to ASNHL
Onset and Subjective Loudness - Safety Population Analysis Set

| | | AM-111 | AM-111 | |
|---|---|---|---|---|
| | | 0.4 | 2.0 | |
| | Placebo | mg/mL | mg/mL | Total |
| | (N = 72) | (N = 68) | (N = 70) | (N = 210) |
| Tinnitus, number | | | | |
| (%) subjects | | | | |
| No | 16 (22.2) | 9 (13.2) | 5 (7.1) | 30 (14.3) |
| Yes | 56 (77.8) | 59 (86.8) | 65 (92.9) | 180 (85.7) |
| Pre-Existing Tinnitus, | 4 (5.6) | 3 (4.4) | 6 (8.6) | 13 (6.2) |
| number (%) subjects | | | | |
| ASNHL Related | 52 (73.2) | 56 (82.4) | 59 (84.3) | 167 (79.9) |
| Tinnitus, | | | | |
| number (%) subjects | | | | |
| Concurrent with ASNHL | 50 (70.4) | 55 (80.9) | 54 (77.1) | 159 (76.1) |
| Within 24 hours | 2 (2.8) | 0 | 5 (7.1) | 7 (3.3) |
| after ASNHL | | | | |
| Within 48 hours | 0 | 1 (1.5) | 0 | 1 (0.5) |
| after ASNHL^{a} | | | | |
| Tinnitus Loudness | | | | |
| (ASNHL Related | | | | |
| Tinnitus) | | | | |
| Mean (SD) | 5.6 (1.9) | 5.4 (2.3) | 5.2 (2.2) | 5.4 (2.2) |
| Median | 6.0 | 5.0 | 5.0 | 5.0 |
| Range | 2 to 10 | 1 to 10 | 1 to 10 | 1 to 10 |

N = number of subjects;
ASNHL = acute sensorineural hearing loss;
SD = standard deviation
^{a}Excluding where tinnitus was within 24 hours after ASNHL.

Incidence of ASNHL-related tinnitus decreased most quickly and most markedly for the AM-111 0.4 mg/mL group (38.0% of affected subjects had ASNHL related tinnitus at D90), followed by the AM-111 2.0 mg/mL group (51.9%) and the placebo group (56.0%). See Table below.

Summary of Frequencies of Tinnitus in Subjects with ASNHL-
Related Tinnitus - Safety Population Analysis Set

| | Number of Subjects with Tinnitus/Number | | | |
|---|---|---|---|---|
| | of Subjects with Observation (%) | | | |
| | | AM-111 | | |
| | | 0.4 | 2.0 | |
| | Placebo | mg/mL | mg/mL | Total |
| Day 0, | 52/52 (100) | 56/56 (100) | 59/59 (100) | 115/115 (100) |
| before | | | | |
| study drug | | | | |
| Day 0, | 48/52 (92.3) | 53/56 (94.6) | 58/59 (98.3) | 111/115 (96.5) |
| after | | | | |
| study drug | | | | |
| Day 3 | 49/52 (94.2) | 51/56 (91.1) | 59/59 (100) | 110/115 (95.7) |
| Day 7 | 45/52 (86.5) | 42/55 (76.4) | 51/58 (87.9) | 93/113 (82.3) |
| Day 30 | 32/52 (61.5) | 26/55 (47.3) | 35/56 (62.5) | 61/111 (55.0) |
| Day 90 | 28/50 (56.0) | 19/50 (38.0) | 28/54 (51.9) | 47/104 (45.2) |

Analysis of PTA improvement by hearing loss severity (Jerger et al., 1980) revealed unexpectedly strong spontaneous recovery for lesser severities: by Day 7, placebo-treated patients enrolling with mild to moderate hearing loss (PTA <60 dB; n=41) had recovered already 28.9 dB or 77% of their initial loss, whilst for patients with severe or profound hearing loss (PTA ≧60 dB; n=30) it was only 17.3 dB or 24%. ANCOVA revealed a statistically significant interaction term between treatment group and hearing loss severity subgroup (p=0.04), indicating that the latter should be analyzed separately. Mild to moderate hearing loss was essentially fully recovered by Day 90 (just 3 dB or 8.1% remained on average).

In the “severe to profound hearing loss” subgroup, a statistically significantly higher percentage of subjects in the AM-111 0.4 mg/mL group achieved complete tinnitus remission over the 90 days compared with placebo (56.0% vs. 26.1%, p=0.045). A higher percentage of subjects in the AM-111 2.0 mg/mL group also achieved complete tinnitus remission compared with placebo, although this did not reach statistical significance (48.3% vs. 26.1%, p=0.152). In the subgroup “mild to moderate hearing loss”, no statistically significant differences were observed between AM-111 treatment groups and placebo. The results of the efficacy of AM-111 on tinnitus in each of the two hearing loss subgroups are summarized in table below.

Frequencies of Subjects with Complete ASNHL-Related Tinnitus
Remission in the Treated Ear, by Treatment Group and
Combined Severity Subgroups - mITT Analysis Set

| Complete Tinnitus Remission | Frequency | Frequency AM-111 | |
|---|---|---|---|
| Time point: Overall | Placebo | 0.4 mg/mL | 2.0 mg/mL |
| All Subjects | N = 51 | N = 50 | N = 56 |
| Frequency (%) | 28 | 35 (70.0%) | 31 (55.4%) |
| p-value placebo - AM-111 | (54.9%) | 0.151 | 1.000 |
| Severe to Profound Subgroup | N = 23 | N = 25 | N = 29 |
| Frequency (%) | 6 (26.1%) | 14 (56.0%) | 14 (48.3%) |
| p-value placebo - AM-111 | | 0.045 | 0.152 |
| Mild to Moderate Subgroup | N = 28 | N = 25 | N = 27 |
| Frequency (%) | 22 | 21 (84.0%) | 17 (63.0%) |
| p-value placebo - AM-111 | (78.6%) | 0.732 | 0.245 |

Comparison of frequency between placebo and active treated groups with Fisher's Exact Test.

The results of the study show that AM-111 appears to be a promising novel approach for treating ASNHL-related tinnitus with a short local therapy, particularly in patients suffering from tinnitus associated with severe to profound acute hearing loss.

All publications, patents and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

### REFERENCES

Each of the references below is herein incorporated by reference in its entirety for all purposes.
Abi-Hachem R N, Zine A, Van De Water T R (2010), The injured cochlea as a target for inflammatory processes, initiation of cell death pathways and application of related otoprotectives strategies. Recent Pat CNS Drug Discov 5(2):147-63.
Anne S, Kisley L B, Tajuddin S T, Leahy P, Alagramam K N, Megerian C A (2007), Molecular changes associated with the endolymphatic hydrops model, Otology & Neurotology 28(6):834-41.
Arbusow V, Schulz P, Strupp M, Dieterich M, von Reinhardstoettner A, Rauch E, Brandt T (1999), Distribution of herpes simplex virus type 1 in human geniculate and vestibular ganglia: implications for vestibular neuritis, Ann Neurol 46: 416-419.
Arnold W, Niedermeyer H P (1997), Herpes simplex virus antibodies in the perilymph of patients with Meniére disease, Arch Otolaryngol Head Neck Surg 123(1):53-6.
Barkdull G C, Hondarrague Y, Meyer T, Harris J P, Keithley E M (2007), AM-111 reduces hearing loss in a guinea pig model of acute labyrinthitis. Laryngoscope 117:2174-82
Beckham J D, Goody R J, Clarke P, Bonny C, Tyler K L (2007), Novel strategy for treatment of viral central nervous system infection by using a cell-permeating inhibitor of c-Jun N-terminal kinase, Journal of Virology 81(13): 6984-6992.
Beyea J A, Agrawal S K, Parnes L S (2012), Recent advances in viral inner ear disorders, Curr Opin Otolaryngol Head Neck Surg 20(5): 404-408.
Bixenstine P J, Maniglia M P, Vasanji A, Alagramam K N, Megerian C A (2008), Spiral ganglion degeneration patterns in endolymphatic hydrops, Laryngoscope 118(7): 1217-1223.
Bogoyevitch et al., (2004), Targeting the JNK MAPK cascade for inhibition: basic science and therapeutic potential. Biochim. Biophys. Acta 1697, 89-101.
Canlon B, Meltser I, Johansson P, Tahera Y. (2007) Glucocorticoid receptors modulate auditory sensitivity to acoustic trauma. Hear Res, 226(1-2), 61-9.
Coleman J K, Littlesunday C, Jackson R, Meyer T (2007), AM-111 protects against permanent hearing loss from acute acoustic trauma. Hear Res 226:70-8.
Derebery M J, Fisher L M, Iqbal Z (2004), Randomized double-blinded, placebo-controlled clinical trial of famciclovir for reduction of Meniére's disease symptoms, Otolaryngol Head Neck Surg 131(6): 877-884.
Derebery J, Berliner K I (2005), Characteristics, onset and progression of Meniére's Disease, 5th International Symposium Meniére's Disease & Inner Ear Homeostasis Disorders poster 0-8.
Eshraghi A A, Gupta C, Van De Water T R, Bohorquez J E, Garnham C, Bas E, Talamo V M (2013), Molecular mechanisms involved in cochlear implantation trauma and the protection of hearing and auditory sensory cells by inhibition of c-Jun-N-terminal kinase signalling. Laryngoscope 123(Suppl 1):S1-514.
Figueiredo R R, Azevedo A A, Oliveira P M. Correlation analysis of the visual-analogue scale and the Tinnitus Handicap Inventory in tinnitus patients. Revista Brasileira de Otorrinolaringologia 75(1):76-9, 2009.
Fishman J M, Burgess C, Waddell A (2011), Corticosteroids for the treatment of idiopathic acute vestibular dysfunction (vestibular neuritis), Cochrane Database Syst Rev 5: CD008607.
Foster C A, Breeze R E (2013), The Meniere attack: an ischemia/reperfusion disorder of inner ear sensory tissues, Medical Hypotheses 81:1108-1115.
Gacek R R, Gacek M R (2002), Meniére's Disease: a form of vestibular ganglionitis, in: Gacek
R R, Gacek M R, eds. Viral neuropathis in the temporal bone. Adv Otorhinolaryngol 60. Basel, Karger: 67-79.
Gacek R R (2009), Meniére's disease is a viral neuropathy, ORL J Otorhinolaryngol Relat Spec 71(2): 78-86.
Garrington and Johnson (1999), Organization and regulation of mitogen-activated protein kinase signaling pathways, Curr Opin Cell Biol. 1999 April; 11(2):211-8.
Gartner M, Bossart W, Linder T (2008), Herpes virus and Meniére's disease, ORL J Otorhinolaryngol Relat Spec 70(1): 28-31.
Gates G (2007), The epidemiology of Meniére's Disease and the problems of diagnosis, 30th Association for Research in Otolargyngology Midwinter research meeting, poster 220.
Grindal T C, Sampson E M, Antonelli P J (2010), AM-111 prevents hearing loss from semicircular canal injury in otitis media. Laryngoscope 120:178-82.
Guyot J P, Maire R, Delaspre O (2008), Intratympanic application of an antiviral agent for the treatment of Meniére's disease, ORL J Otorhinolaryngol Relat Spec 70(1): 21-6.
Hargett D, McLean T, Bachenheimer S L (2005), Herpes simplex virus ICP27 activation of stress kinases JNK and p38, Journal of Virology 79(13): 8348-8360.
Hu B H, Henderson D, Nicotera T M (2002), Involvement of apoptosis in progression of cochlear lesion following exposure to intense noise, Hear Res 166:62-71.
Huy P T, Sauvaget E. Idiopathic sudden sensorineural hearing loss is not an otologic emergency. Otol Neurotol 26(5):896-902, 2005.
Kamalski D M, Hoekstra C E, van Zanten B G, et al. Measuring disease-specific health-related quality of life to evaluate treatment outcomes in tinnitus patients: a systematic review. Otolaryngol Head Neck Surg 143(2):181-5, 2010.
Labbe D, Teranishi M, Hess A, Bloch W, Michel 0 (2005), Activation of caspase-3 is associated with oxidative stress in the hydropic guinea pig cochlea, Hearing Research 202:21-27.
Lambert P R, Nguyen S, Maxwell K S, Tucci D L, Lustig L R, Fletcher M, Bear M, Lebel C (2012), A randomized, double-blind, placebo-controlled clinical study to assess safety and clinical activity of OTO-104 given as a single intratympanic injection in patients with unilateral Meniére's disease, Otol Neurotol 33(7): 1257-1265.
Linthicum F (2001), Herpes simplex virus DNA in endolymphatic sacs in patients with Meniere's disease, The National Temporal Bone Registry Newsletter 9, #2.
Ma C, Billings P, Harris J P et al. (2000), Characterization of an experimentally induced inner ear immune response, Laryngoscope 110:451-56.
Manning and Davis (2003), Targeting JNK for therapeutic benefit: from junk to gold? Nature Reviews Drug Discovery 2, 554-565
McLean T I, Bachenheimer S L (1999), Activation of cJUN N-Terminal kinase by herpes simplex virus type 1 enhances viral replication, Journal of Virology 73(10): 8415-8426.
Meikle M B, Henry J A, Griest S E, et al. The Tinnitus Functional Index: development of a new clinical measure for chronic, intrusive tinnitus. Ear Hear 33(2):153-76, 2012.
Merchant S N, Adams J C, Nadol J B (2005), Pathophysiology of Meniere's syndrome: are symptoms caused by endolymphatic hydrops? Otol Neurotol 26(1):74-81.
Mikulec A A, Plontke S K, Hartsock J J, Salt A N (2009), Entry of substances into perilymph through the bone of the otic capsule after intratympanic applications in guinea pigs: implications for local drug delivery in humans, Otol Neurotol 30(2):131-8.
Omotehara Y, Hakuba N, Hato N, Okada M, Gyo K (2011), Protection against ischemic cochlear damage by intratympanic administration of AM-111. Otol & Neurotol 32(9):1422-7.
Plontke S K, Mynatt R, Gill R M, Borgmann S, Salt A N (2007), Concentration gradient along the scala tympani after local application of gentamicin to the round window membrane, Laryngoscope 117: 1191-8.
Pyykko I, Zou J (2008), Do viruses cause inner ear disturbances? ORL J Otorhinolaryngol Relat Spec 70(1): 32-40.
Rask-Andersen H1, Friberg U, Johansson M, Stjernschantz J (2005), Effects of intratympanic injection of latanoprost in Meniere's disease: a randomized, placebo-controlled, double-blind, pilot study, Otolaryngol Head Neck Surg 133(3): 441-3.
Salt A N, Plontke S N (2009), Principles of local drug delivery to the inner ear, Audio′ Neurotol 14: 350-360.
Semaan M T, Alagramam K N, Megerian C A (2005), The basic science of Meniére's disease and endolymphatic hydrops, Current Opinion in Otolaryngology & Head and Neck Surgery 13:301-307.
Strupp M, Zingler V C, Arbusow V, Niklas D, Maag K P, Dieterich M, Bense S, Theil D, Jahn K, Brandt T (2004), Methylprednisolone, valacyclovir, or the combination for vestibular neuritis, New England Journal of Medicine 351: 354-361.
Strupp M, Brandt T (2013), Peripheral vestibular disorders, Curr Opin Neurol 26(1): 81-9.
Strupp M, Brandt T (2009), Vestibular neuritis, Semin Neurol 29(5):509-19.
Vrabec J T (2003), Herpes simplex virus and Meniére's Disease, Laryngoscope 113:1431-1438.
Yazawa Y, Suzuki M, Hanamitsu M, Kimura H, Tooyama I (2003), Detection of viral DNA in the endolymphatic sac in Meniére's disease by in situ hybridization, ORL J Otorhinolaryngol Relat Spec 65(3): 162-168.
Wang J, Van De Water T R, Bonny C, de Ribaupierre F, Puel J L, Zine A (2003), A peptide inhibitor of c-Jun N-terminal kinase (D-JNKI-1) protects against both aminoglycoside and acoustic trauma-induced auditory hair cell death and hearing loss. J Neurosci 23:8596-607.
Wang J, Ruel J, Ladrech S, Bonny C, Van de Water T R, Puel J L (2007), Inhibition of the JNK-mediated mitochondrial cell death pathway restores auditory function in sound exposed animals, Mol Pharmacol 71 (3): 654-666.
Wei L, Zhu Z, Wang J, Liu J (2009), JNK and p38 mitogen-activated protein kinase pathways contribute to porcine circovirus type 2 infection, J Virol 83(12):6039
Welling D B, Miles B A, Western L, Prior T W. (1997), Detection of viral DNA in vestibular ganglia tissue from patients with Meniere's disease. Am J. Otol 18(6) 734-7,
Zine A, Van de Water T R (2004), The MAPK/JNK signalling pathway offers potential therapeutic targets for the prevention of acquired deafness. Curr Drug Targets CNS Neurol Disord 3(4):325-32.

1. A method of treating Meniére's Disease in a human in need thereof comprising administering to the human a pharmaceutical composition comprising a therapeutically effective amount of a peptide inhibitor of c-Jun N-terminal kinase (JNK) or a pharmaceutically acceptable salt thereof.

2. The method of claim 2, wherein the peptide inhibitor is no more than 50 amino acids in length and comprises a sequence that is at least 80% identical to the sequence of any one of SEQ ID NOs: 1 to 4 and 13 to 45.

3. The method of claim 2, wherein the peptide inhibitor comprises a sequence that is at least 90% identical to DQSRPVQPFLNLTTPRKPR (SEQ ID NO: 1) or RPKRPTTLNLFPQVPRSQD (SEQ ID NO: 4).

4. The method of claim 1, wherein the peptide inhibitor comprises or consists of the sequence of DQSRPVQPFLNLTTPRKPRPPRRRQRRKKRG (SEQ ID NO: 2) or GRKKRRQRRRPPRPKRPTTLNLFPQVPRSQD (SEQ ID NO: 3).

5. The method of claim 1, wherein all of the chiral amino acids in the peptide inhibitor are in the D configuration.

6. The method of claim 1, wherein all of the chiral amino acids in the peptide inhibitor are in the L configuration.

7. The method of claim 1, wherein the pharmaceutical composition reduces the severity of at least two symptoms of the Meniére's Disease.

8. The method of claim 7, wherein the symptom is selected from the group consisting of vertigo, dizziness, tinnitus, hearing loss and the sensation of pressure or pain in the affected ear.

9. The method of claim 1, wherein the symptoms of Meniére's Disease results from idiopathic endolymphatic hydrops.

10. The method of claim 1, wherein the pharmaceutical composition is administered locally to the round window membrane or oval window of the ear.

11. The method of claim 1, wherein the pharmaceutical composition is administered by an intratympanic injection.

12. The method of claim 1, wherein the pharmaceutical composition is administered systemically.

13. The method of claim 1, wherein a single dose or repeated doses are administered to the human whenever there is an attack of one or more symptoms of Meniére's Disease.

14. The method of claim 1, wherein the pharmaceutical composition is administered to the human within about 48 to 72 hours following an attack of one or more symptoms of Meniére's Disease.

15. The method of claim 1, wherein the human has, or is diagnosed with, or has risk of progressive hearing loss.

16. The method of claim 1, wherein the human has, or is diagnosed with, or has risk of persisting tinnitus.

17. The method of claim 1, wherein the pharmaceutical composition is a gel.

18. The method of claim 1, wherein the pharmaceutical composition is an implant.

19. The method of claim 1, wherein the pharmaceutical composition is used for recurrent treatment of Meniére's Disease.

20. The method of claim 1, wherein the pharmaceutical composition is used for continuous treatment of Meniére's Disease.

21. The method of claim 1, wherein the pharmaceutical composition comprises about 0.5% to about 2% of hyaluronic acid.

22. The method of claim 1, wherein the pharmaceutical composition comprises a phosphate buffer which buffers the pH of the composition to 6.0 to 7.4.

23. The method of claim 1, wherein the pharmaceutical composition comprises about 1 to 500 μM of a peptide inhibitor of c-Jun N-terminal kinase (JNK) or a pharmaceutically acceptable salt thereof.

24. The method of claim 1, wherein the pharmaceutical composition is administered in multiple doses.

25. The method of claim 1, wherein the pharmaceutical composition is combined with one or more other treatments.

26. The method of claim 25, wherein the other treatment comprising administration of an antiviral, diuretic, and/or antiemetic agent.

27. The method of claim 1, wherein the administration provides statistically significant therapeutic effect for the treatment of Meniére's Disease.

28. A method of attenuating long-term outcomes of Meniére's Disease in a human in need thereof comprising administering to the human a pharmaceutical composition comprising a therapeutically effective amount of a peptide inhibitor of c-Jun N-terminal kinase (JNK) or a pharmaceutically acceptable salt thereof.

29. The method of claim 28, wherein the long-term outcomes include progressive hearing loss.

30. The method of claim 28, wherein the long-term outcomes include persisting tinnitus.
